(19) United States
(12) Patent Application Publication
(10) Pub. No.: US 2019/0247342 A1
(43) Pub. Date: Aug. 15, 2019

(54) Alpha-Ketobutyrate, 2-Hydroxybutyrate, and Alpha-Ketoglutarate for Stimulating Hair Growth

(71) Applicant: The Regents of the University of California, Oakland, CA (US)

(72) Inventors: Jing Huang, Los Angeles, CA (US); Min Chai, Los Angeles, CA (US); Meisheng Jiang, Thousand Oaks, CA (US); Xudong Fu, Los Angeles, CA (US); Heejun Hwang, Los Angeles, CA (US)

(21) Appl. No.: 16/332,763

(22) PCT Filed: Sep. 29, 2017
(86) PCT No.: PCT/US2017/054282

§ 371 Date: Mar. 12, 2019

Related U.S. Application Data
(60) Provisional application No. 62/402,543, filed on Sep. 30, 2016.

(51) Int. Cl. A61K 31/19; A61K 31/194; A61Q 7/00

(52) U.S. Cl. CPC A61K 31/19; A61Q 7/00; A61K 31/194

(57) ABSTRACT

Disclosed herein are methods and compositions for treating, inhibiting, or reducing hair loss, treating, inhibiting, or reducing pigmentation loss, improving or stimulating hair growth, and/or improving or stimulating pigmentation production in a subject with one or more alpha-ketobutyrate compounds and/or one or more glutarate compounds.

### ACKNOWLEDGEMENT OF GOVERNMENT SUPPORT

This invention was made with Government support under Grant Number R01 AT006889, awarded by the National Institutes of Health. The Government has certain rights in the invention.

### BACKGROUND OF THE INVENTION

Hair originates in a deep pouch-like structure in the epidermis—known as the hair follicle—which penetrates the dermis. A hair root extends down in the hair follicle and widens into an indented bulb at its base. Newly dividing cells at the base of the hair multiply, forcing cells above them upward. As cells move upward, they gradually die and harden into a hair shaft.

### SUMMARY OF THE INVENTION

Disclosed herein are compositions and methods of stimulating new hair growth.

In some embodiments, the present invention is directed to a method of stimulating new hair growth in a subject in need thereof, which comprises administering to the subject a pharmaceutical composition that comprises an α-ketobutyrate compound and/or a glutarate compound described herein. In some embodiments, the present invention is directed to a dosage form that comprises an α-ketobutyrate compound and/or a glutarate compound as described herein. In some embodiments, the present invention is directed to a topical pharmaceutical composition that comprises an α-ketobutyrate compound and/or a glutarate compound as described herein.

In some embodiments, the present invention is directed to a method for treating, inhibiting, or reducing hair loss in a subject which comprises administering to the subject a therapeutically effective amount of one or more one or more α-ketobutyrate compounds and/or one or more glutarate compounds. In some embodiments, the present invention is directed to a method for improving or stimulating hair growth in a subject which comprises administering to the subject a therapeutically effective amount of one or more α-ketobutyrate compounds and/or one or more glutarate compounds.

In some embodiments, the present invention is directed to a method for treating, inhibiting, or reducing pigmentation loss in a subject which comprises administering to the subject a therapeutically effective amount of one or more α-ketobutyrate compounds and/or one or more glutarate compounds. In some embodiments, the present invention is directed to a method for improving or stimulating pigmentation production in a subject which comprises administering to the subject a therapeutically effective amount of one or more α-ketobutyrate compounds and/or one or more glutarate compounds. In some embodiments, the hair loss is a result of the subject aging. In some embodiments, the pigmentation loss is a result of the subject aging. In some embodiments, the subject is aging and/or the subject is an aged subject. In some embodiments, the therapeutically effective amount is administered as several doses over a given period of time, e.g., a daily dose for a week or more. In some embodiments, the therapeutically effective amount is administered as a daily dose of about 0.01-1.0, preferably about 0.01-0.5, more preferably about 0.1-0.2 grams per kilogram body weight per day. In some embodiments, about 0.05 to about 2 grams of the one or more α-ketobutyrate compounds and/or the one or more glutarate compounds per kilogram weight of the subject is administered to the subject daily for at least a week. In some embodiments, the one or more α-ketobutyrate compounds is α-ketobutyrate (α-KB), the one or more glutarate compounds is alpha-ketoglutarate (α-KG), and/or the one or more glutarate compounds is 2-hydroxypentanedioate (2-HG). In some embodiments, the present invention is directed to an α-ketobutyrate compound and/or a glutarate compound for use in treating, inhibiting, or reducing hair loss, treating, inhibiting, or reducing pigmentation loss, improving, or stimulating hair growth, and/or improving or stimulating pigmentation production in a subject.

In some embodiments, the present invention is directed to a method of stimulating new hair growth in a subject in need thereof, which comprises administering to the subject a pharmaceutical composition comprising:
a therapeutically effective amount of a compound of Formula I:

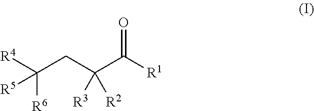

wherein:
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;
or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof; and
an excipient.

In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is —OR⁷, wherein R⁷ is C₁₋₂₀ substituted or unsubstituted alkyl. In some embodiments, R² is hydrogen, halogen, —CN, —CHO, or —NR⁸R⁹, wherein R⁸ and R⁹ are each independently hydrogen or substituted or unsubstituted alkyl. In some embodiments, R² and R³, together with the atom to which they are bound, form an oxo. In some embodiments, R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl. In some embodiments, R⁴ is —COOR⁷ or —CONR⁸R⁹, wherein R⁷, Re, and R⁹ are each independently hydrogen or C₁₋₂₀ substituted or unsubstituted alkyl.

In some embodiments, the pharmaceutical composition is administered to an area on the subject where new hair growth is desired. In some embodiments, the area has an amount of hair that is less than the amount present at an earlier period. In some embodiments, the area is absent of hair. In some embodiments, the area is absent of hair due to a disease or condition that decreases or inhibits hair growth. In some embodiments, the area is absent of hair due to an injury. In some embodiments, the area is absent of hair due to chemotherapy and/or radiation therapy. In some embodiments, the area is absent of hair due to surgery.

In some embodiments, the subject has a thyroid disorder. In some embodiments, the subject has a pituitary gland disorder. In some embodiments, the subject has alopecia areata. In some embodiments, the subject has anagen effluvium and/or telogen effluvium.

In some embodiments, the compound of Formula I is alpha-ketoglutarate (α-KG). In some embodiments, the compound of Formula I is 2-HB. In some embodiments, the compound of Formula I is alpha-ketobutyrate (α-KB). In some embodiments, the concentration of α-KG present in the pharmaceutical composition is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KG present in the pharmaceutical composition is about 16 mM. In some embodiments, the concentration of α-KB present in the pharmaceutical composition is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KB present in the pharmaceutical composition is about 16 mM. In some embodiments, the concentration of 2-HB present in the pharmaceutical composition is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of 2-HB present in the pharmaceutical composition is about 16 mM.

In some embodiments, the pharmaceutical composition is formulated for oral, parenteral, or topical administration. In some embodiments, the pharmaceutical composition is formulated for topical administration. In some embodiments, the pharmaceutical composition is formulated as a gel. In some embodiments, the pharmaceutical composition is formulated as a cream. In some embodiments, the pharmaceutical composition is formulated as an ointment. In some embodiments, the pharmaceutical composition is formulated as a paste. In some embodiments, the pharmaceutical composition is formulated as a lotion.

In some embodiments, the therapeutically effective amount is administered as a single dose. In some embodiments, the therapeutically effective amount is administered in at least two doses, at least three doses, at least four doses, at least five doses, or more. In some embodiments, the therapeutically effective amount is administered daily. In some embodiments, the therapeutically effective amount is administered every other day.

In some embodiments, the method further comprises administering to the subject an additional agent. In some embodiments, the additional agent comprises one or more growth factors. In some embodiments, the growth factor comprises TGF-32, IGF-1, KGF, or HGF. In some embodiments, the additional agent is administered in combination with the pharmaceutical composition. In some embodiments, the additional agent is administered sequentially with the pharmaceutical composition. In some embodiments, the additional agent and the pharmaceutical composition are administered as a unified dosage form. In some embodiments, the additional agent and the pharmaceutical composition are administered as separate dosage forms.

In some embodiments, the number of hair follicles in the subject after administration of the pharmaceutical composition is higher relative to the number of hair follicles in the subject prior to administration of the pharmaceutical composition. In some embodiments, the weight of a hair in the subject after administration of the pharmaceutical composition is greater relative to the weight of a hair in the subject prior to administration of the pharmaceutical composition. In some embodiments, the hair shaft length of a hair in the subject is increased faster after administration of the pharmaceutical composition relative to the hair shaft length of a hair in the subject prior to administration of the pharmaceutical composition. In some embodiments, the growth rate of a hair in the subject is increased after administration of the pharmaceutical composition relative to the growth rate of a hair in the subject prior to administration of the pharmaceutical composition. In some embodiments, the subject is a human.

In some embodiments, the present invention is directed to a dosage form comprising a compound of Formula I:

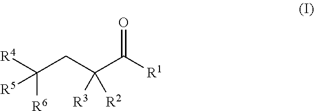

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;
or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof; and an excipient.

In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is —OR⁷, wherein R⁷ is C₁₋₂₀ substituted or unsubstituted alkyl. In some embodiments, R² is hydrogen, halogen, —CN, —CHO, or —NR⁸R⁹, wherein R⁸ and R⁹ are each independently hydrogen or substituted or unsubstituted alkyl. In some embodiments, R² and R³, together with the atom to which they are bound, form an oxo. In some embodiments, R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl. In some embodiments, R⁴ is —COOR⁷ or —CONR⁸R⁹, wherein R⁷, Re, and R⁹ are each independently hydrogen or C₁₋₂₀ substituted or unsubstituted alkyl.

In some embodiments, the dosage form is formulated for stimulating a cell to enter into anagen phase. In some embodiments, the compound of Formula I is alpha-ketoglutarate (α-KG). In some embodiments, the compound of Formula I is 2-hydroxybutyrate (2-HB). In some embodiments, the compound of Formula I is alpha-ketobutyrate (α-KB). In some embodiments, the concentration of α-KG is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KG is about 16 mM. In some embodiments, the concentration of α-KB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KB is about 16 mM. In some embodiments, the concentration of 2-HB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the dosage form is formulated for oral, parenteral, or topical administration. In some embodiments, the dosage form is formulated for topical administration. In some embodiments, the dosage form is formulated as a gel. In some embodiments, the dosage form is formulated as a cream. In some embodiments, the dosage form is formulated as an ointment. In some embodiments, the dosage form is formulated as a paste. In some embodiments, the dosage form is formulated as a lotion. In some embodiments, the dosage form is administered as a single dose. In some embodiments, the dosage form is administered in at least two doses, at least three doses, at least four doses, at least five doses, or more. In some embodiments, the dosage form is administered daily. In some embodiments, the dosage form is administered every other day. In some embodiments, the dosage form further comprises an additional agent. In some embodiments, the additional agent comprises one or more growth factors. In some embodiments, the growth factor comprises TGF-β2, IGF-1, KGF, or HGF. In some embodiments, the additional agent is administered in combination with the dosage form. In some embodiments, the additional agent is administered sequentially with the dosage form.

In some embodiments, the present invention is directed to a topical pharmaceutical composition comprising a compound of Formula I:

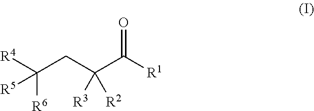

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof, and
a tissue penetrating enhancer.

In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is —OR⁷, wherein R⁷ is C₁₋₂₀ substituted or unsubstituted alkyl. In some embodiments, R² is hydrogen, halogen, —CN, —CHO, or —NR⁸R⁹, wherein R⁸ and R⁹ are each independently hydrogen or substituted or unsubstituted alkyl. In some embodiments, R² and R³, together with the atom to which they are bound, form an oxo. In some embodiments, R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl. In some embodiments, R⁴ is —COOR⁷ or —CONR⁸R⁹, wherein R⁷, Re, and R⁹ are each independently hydrogen or C₁₋₂₀ substituted or unsubstituted alkyl. In some embodiments, the compound of Formula I is alpha-ketoglutarate (α-KG). In some embodiments, the compound of Formula I is 2-hydroxybutyrate (2-HB). In some embodiments, the compound of Formula I is alpha-ketobutyrate (α-KB). In some embodiments, the concentration of α-KG is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KG is about 16 mM. In some embodiments, the concentration of α-KB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the concentration of α-KB is about 16 mM. In some embodiments, the concentration of 2-HB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM. In some embodiments, the topical pharmaceutical composition is formulated as a gel. In some embodiments, the topical pharmaceutical composition is formulated as a cream. In some embodiments, the topical pharmaceutical composition is formulated as an ointment. In some embodiments, the topical pharmaceutical composition is formulated as a paste. In some embodiments, the topical pharmaceutical composition is formulated as a lotion.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE DRAWINGS

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

This invention is further understood by reference to the drawings wherein:

FIG. 1 schematically shows the experimental protocol. Mice were topically (unless indicated otherwise) treated every other day with the indicated amount and photos were taken every week. Mice were monitored for the appearance of skin pigmentation, signaling the initiation of anagen. No hair growth (and no pigmentation) was assigned the arbitrary value of 0. Skin darkening was given a value from 0 to 100%, with the higher number indicating darker skin and visible hair growth.

FIG. 2 to FIG. 6. Administration of α-KB delays aging and promotes hair growth. FIG. 2 is a graph showing that α-KB extends the lifespan of adult worms, mean lifespan (days of adulthood) with vehicle treatment (mᵥₑₕ)=14.1 (n=111 animals tested), m_{α-KB}=22.4 (n=66), P<0.0001 (log-rank test). From left to right, the first line is vehicle. FIG. 3 is a graph showing that α-KB increases the lifespan of old male C57BL/6J mice (*P=0.0476, by Fisher's exact test, two-tailed), but not old female mice. FIG. 4 is a picture showing that administration of α-KB delays or reduces age-related hair loss in mice compared to negative controls. Mice were not shaved prior to treatment. FIG. 5 is a graph showing that administration of α-KG reduces age-related pigmentation loss. From left to right, the first line is α-KB. FIG. 6 is a picture evidencing that treatment with α-KB improved pigmentation and stimulates hair growth by Day 14 compared to negative controls.

FIG. 7 to FIG. 9. Administration of 2-HB delays aging and promotes hair growth. FIG. 7 is a graph showing that 2-HB extends the lifespan of adult worms, mean lifespan (days of adulthood) with vehicle treatment (mᵥₑₕ)=15.4 (n=109 animals tested), m_{2.HB}=19.8 (n=98), P<0.0001 (log-rank test). From left to right, the first line is vehicle. FIG. 8 is a graph evidencing that pigmentation improved in mice treated with 2-HB compared to negative controls. From left to right, the first line is 2-HB. FIG. 9 is a picture evidencing that treatment with 2-HB accelerated pigmentation and hair growth on Day 14 compared to negative controls.

FIG. 10 to FIG. 13. Administration of α-KG delays aging and promotes hair growth. FIG. 10 is a graph showing that α-KG extends the lifespan of adult worms, mean lifespan (days of adulthood) with vehicle treatment (mᵥₑₕ)=16.3 (n=100 animals tested), m_{α-KG}=26.1 (n=104), P<0.0001 (log-rank test). FIG. 11 is a graph showing that treatment with α-KG improves hair pigmentation compared to negative controls. From left to right, the first line is α-KG. FIG. 12 is a picture evidencing that treatment with α-KG improves pigmentation and stimulates hair growth by Day 14. FIG. 13 is a picture evidencing that oral administration of α-KG significantly induces hair growth compared to negative controls. Hair growth at Week 10 is shown.

### DETAILED DESCRIPTION OF THE INVENTION

As disclosed herein, α-KB, α-KG, and 2-HG reduced or inhibited hair loss, stimulated hair growth, and/or improved pigmentation in aging subjects.

In some instances, hair cycles are divided into three stages: 1) anagen, the active growing phase of the hair follicle cycle, 2) catagen, a regressive stage when the follicle begins to become dormant, and 3) telogen, the resting or dormant stage lasting 3 to 4 months. When the dormant phase ends, an old hair falls out. A hair follicle then returns to the anagen phase and a new hair begins to grow.

In some instances, the niche of a mammalian hair follicle comprises a heterogeneous cell population, which comprises hair follicle stem cells (HFSCs) and epithelial cells keratinocytes and melanocytes. In some instances, HFSCs and epithelial cells further interact with mesenchymal lineage dermal papilla cells (DPCs) embedded in the hair bulb, and dermal cells such as fibroblasts, immune cells, and adipocytes.

During the hair cycle, multiple signaling factors such as Wnt/β-catenin, sonic hedgehog (SHH), bone morphogenetic protein (BMP), transforming growth factor-β (TGF-β), and Notch; transcription factor such as Fork-head box C1 (FOXC1); and paracrine factors such as growth factors modulate and mediate the proliferation of matrix keratinocytes and differentiation of HFSCs or their progenitor cells into mature hair cells. In some cases, dysregulation or disruption of these signaling factors, transcription factor, or paracrine factors result in the loss of hair.

In some embodiments, the present invention is directed to methods for treating, inhibiting, or reducing hair loss, improving or stimulating hair growth, treating, inhibiting, or reducing pigmentation loss, and/or improving or stimulating pigmentation production in a subject which comprises administering the subject one or more α-KB compounds and/or one or more glutarate compounds. In some embodiments, the present invention is directed to compositions for treating, inhibiting, or reducing, improving or stimulating hair growth, treating, inhibiting, or reducing pigmentation loss, and/or improving or stimulating pigmentation production in a subject, said compositions comprise a one or more α-KB compounds and/or one or more glutarate compounds. In some embodiments, the subject is an animal. In some embodiments, the subject is a nematode, a rodent, or a non-human primate. In some embodiments, the subject is a human. In some embodiments, the subject is aging. In some embodiments, the subject is an aged subject.

As used herein, a subject who is “aging” refers to a subject in the period of life when untreated control subjects begin to physically, mentally, and/or biologically deteriorate. In some embodiments, a subject who is aging is one whose chronological age is at least at the median point of the average lifespan of untreated control subjects.

As used herein, an “aged” subject is one whose chronological age is at least two-thirds the average life expectancy of untreated control subjects. For example, if the average life expectancy of a given strain of a laboratory mouse is 2 years, an aged mouse of that strain is at least 16 months, and if the average life expectancy of another strain of laboratory mouse is 3 years, an aged mouse of that strain is 24 months. For humans, if the average life expectancy of a human is about 80 years, an aged human is about 53 years. It should be noted that a subject who is aging may or may not be an aged subject.

### Indications

Disease or Condition that Decrease or Inhibit Hair Growth

Hair loss can be caused by inherited factors, disease, stress, medicines, injury or trauma, aging, or poor hair care. Inherited hair loss—or androgenetic alopecia—trigger a sensitivity to a class of hormones called androgens, including testosterone, which causes the hair follicles to shrink. Shrinking follicles produce thinner hair and eventually none at all. It is also known as male pattern baldness and female pattern baldness. Diseases or conditions causing hair loss include syphilis; cancer; autoimmune disorders such as alopecia areata, lupus, lichen planopilaris, sarcoidosis; hypothyroidism; polycystic ovary syndrome; anemia; or condition such as trichotillomania—a compulsive behavior in which a person pulls hair out of the scalp; eyelashes, or eyebrows. Fungal causes such as seborrheic dermatitis and ringwork (tinea capitis); and bacterial causes such as folliculitis decalvans also lead to hair loss. Changes in hormone levels, for example during pregnancy or due to birth control pills, or menopause, also lead to hair loss.

Other causes of hair loss include emotional, mental or physical stress, such as surgery, illness, or high fever; side effects of medicines or medical treatments, such as blood thinners (anticoagulants), anti-depressants or chemotherapy; injury to scalp including scarring; poor nutrition, for example, lack of protein; excess Vitamin A; Vitamin B deficiency; and dramatic weight loss.

### Injury

Any type of a scalp reaction or injury that results in a lesion that causes a scar can cause hair loss or death of the hair follicles.

Chemotherapy and/or Radiation Therapy

Chemotherapy usually refers to the use of medicines or drugs to treat cancer. Chemotherapy drugs are powerful enough to kill rapidly growing cancer cells, but they can also cause harm to perfectly healthy cells, causing side effects throughout the body. Chemotherapy can cause hair loss by harming the cells that help hair grow.

Radiation therapy is the treatment of disease, especially cancer, using X-rays or similar forms of radiation. Radiation therapy kills cancer cells by damaging their DNA. Since radiation therapy can also kill normal healthy cells, it leads a number of side effects, including hair loss.

### Surgery

Stress is a major factor in surgery-related hair loss. During stress, the body directs nutrients to the heart, lungs, muscles and other vital organs. As a result, hair may be weakened and in some cases, hair follicles stop producing new hair. This is called telogen effluvium. This is the most common type of hair loss and typically seen two to three months after a major body stress. Such as major surgery, chronic illness, or significant infection.

### Thyroid Disorders

Thyroid disorders include both an underactive thyroid gland (hypothyroidism) and an overactive thyroid gland (hyperthyroidism). Hair growth depends on the proper functioning of the thyroid gland, and abnormal levels of thyroid hormone produced by this gland can result in hair changes like hair loss.

### Pituitary Gland Disorders

Pituitary gland disorders are disorders of the pituitary gland resulting in too much or too little of one or more of the several hormones its produces. Disorders of the pituitary gland can cause a variety of symptoms and, in some cases, result in serious complications. Symptoms of pituitary gland problems depend upon the specific hormones that are affected and whether they are present in excess amounts or insufficient amounts. For example, overproduction of thyroid-stimulating hormone (TSH) can cause symptoms of an overactive thyroid gland, including hair loss, feelings of nervousness, racing heartbeat, and weight loss.

### Alopecia Areata

Alopecia areata, also known as spot baldness, is an autoimmune disease in which hair is lost from some or all areas of the body, usually from the scalp due to the body's failure to recognize its own body cells and subsequent destruction of its own tissue. There are two types: (1) scarring alopecia, where there is fibrosis, inflammation, and loss of hair follicles, and (2) non-scarring alopecia, where the hair shafts are gone but the hair follicles are preserved, making this type of alopecia reversible.

### Anagen Effluvium and Telogen Effluvium

Anagen effluvium refers to hair shedding that arises during the anagen or growth stage of the hair cycle. Anagen effluvium occurs after any insult to the hair follicle that impairs its mitotic or metabolic activity. It may lead to diffuse non-scarring alopecia (baldness).

Telogen effluvium, on the other hand, refers to hair shedding that arises during the telogen or the resting stage of the hair cycle. It occurs when some stress causes hair roots to be pushed prematurely into the resting stage. Telogen effluvium can be acute or chronic. A “shock” to the system can result in as many as 70% of the scalp hairs to be shed in large numbers about 2 months after the “shock”.

### Compounds

Compounds according to the present invention include compounds having the following structural Formula I:

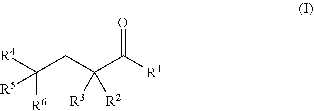

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl; R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo; R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or a salt thereof.

In some embodiments of a compound of Formula I, R¹ is hydrogen, halogen, —CHO, —OR⁷, —NR⁸R⁹, —COOR⁷, —CONR⁸R⁹, or —SR¹⁰. In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is —OR⁷, wherein R⁷ is C1-20 substituted or unsubstituted alkyl.

In some embodiments of a compound of Formula I, R² is hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl and R³ is hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl. In some embodiments, R² is hydrogen, halogen, —CN, —CHO, or —NR⁸R⁹, wherein R⁸ and R⁹ are each independently hydrogen or substituted or unsubstituted alkyl. In some embodiments, R³ is hydrogen, halogen, —CN, —CHO, or —NR⁸R⁹, wherein Re and R⁹ are each independently hydrogen or substituted or unsubstituted alkyl. In some embodiments, R² and R³, together with the atom to which they are bound, form an oxo.

In some embodiments of a compound of Formula I, R⁴ is hydrogen, —CHO, —OR⁷, —NR⁸R⁹, —COOR⁷, or —CONR⁸R⁹. In some embodiments, R⁵ is hydrogen, —CHO, —OR⁷, —NR⁸R⁹, —COOR⁷, or —CONR⁸R⁹. In some embodiments, R⁶ is hydrogen, —CHO, —OR⁷, —NR⁸R⁹, —COOR⁷, or —CONR⁸R⁹.

In some embodiments of a compound of Formula I, R⁷ is hydrogen or C₁₋₂₀ substituted or unsubstituted alkyl. In some embodiments, Re is hydrogen or C₁₋₂₀ substituted or unsubstituted alkyl. In some embodiments, R⁹ is hydrogen or C₁₋₂₀ substituted or unsubstituted alkyl.

In some embodiments, a compound of Formula I is represented by the structure:

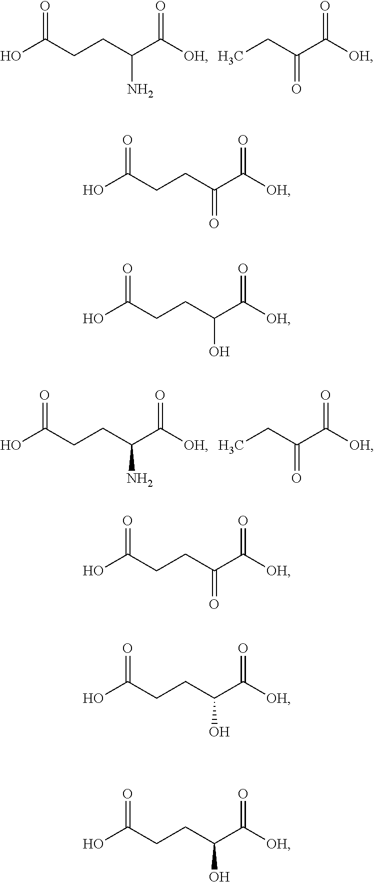

or a salt thereof.

Included within compounds of Formula I are “α-KB compounds” also referred to as “α-ketobutyrate compounds”, which include α-ketobutyrate (α-KB), α-ketobutyric acid, and compounds having the following structural Formula II:

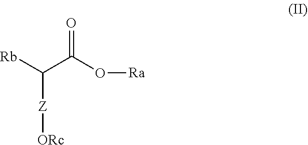

wherein
Ra is a negative charge, H, —CH₃, —CH₂—CH₃, a straight or branched C1-C3 alkyl, a straight or branched C1-C4 alkyl, a straight or branched C1-C5 alkyl, a straight or branched C1-C10 alkyl, —CH₂═CH₃, a straight or branched C1-C3 alkenyl, a straight or branched C1-C4 alkenyl, a straight or branched C1-C5 alkenyl, or a straight or branched C1-C10 alkenyl, Rb is H, —CH₃, —CH₂—CH₃, a straight or branched C1-C3 alkyl, a straight or branched C1-C4 alkyl, a straight or branched C1-C5 alkyl, a straight or branched C1-C10 alkyl, —CH₂═CH₃, a straight or branched C1-C3 alkenyl, a straight or branched C1-C4 alkenyl, a straight or branched C1-C5 alkenyl, or a straight or branched C1-C10 alkenyl,
Rc is optionally present, and if present, Rc is H, —CH₃, —CH₂—CH₃, a straight or branched C1-C3 alkyl, a straight or branched C1-C4 alkyl, a straight or branched C1-C5 alkyl, a straight or branched C1-C10 alkyl, —CH₂═CH₃, a straight or branched C1-C3 alkenyl, a straight or branched C1-C4 alkenyl, a straight or branched C1-C5 alkenyl, or a straight or branched C1-C10 alkenyl, and if absent, Z is a double bond,
and pharmaceutically acceptable solvates, salts, prodrugs, and metabolites thereof.

In some embodiments, Ra is a negative charge, H, or —CH₃. In some embodiments, Rb is H, —CH₃, —CH₂—CH₃, a straight or branched C1-C3 alkyl, —CH₂═CH₃, or a straight or branched C1-C3 alkenyl. In some embodiments, Z is a double bond. In some embodiments, Ra is a negative charge, H, or —CH₃ and Rb is H, —CH₃, —CH₂—CH₃, a straight or branched C1-C3 alkyl, a straight or branched C1-C4 alkyl, a straight or branched C1-C5 alkyl, a straight or branched C1-C10 alkyl, —CH₂═CH₃, a straight or branched C1-C3 alkenyl, a straight or branched C1-C4 alkenyl, a straight or branched C1-C5 alkenyl, or a straight or branched C1-C10 alkenyl. In some embodiments, Ra is a negative charge, H, or —CH₃ and Rb is H, —CH₃, —CH₂—CH₃, a straight or branched C1-C3 alkyl, —CH₂═CH₃, or a straight or branched C1-C3 alkenyl. In some embodiments, Ra is a negative charge, H, or —CH₃, Rb is H, —CH₃, —CH₂—CH₃, a straight or branched C1-C3 alkyl, —CH₂═CH₃, or a straight or branched C1-C3 alkenyl, and Z is a double bond.

α-KB compounds also include various species specific analogues. For example, unless explicitly specified as being of a particular species, “α-KB” includes human α-ketobutyrate, porcine α-ketobutyrate, murine α-ketobutyrate, bovine α-ketobutyrate, and the like. As used herein, the abbreviation “KB” may be used to refer to the term “ketobutyrate”, e.g., α-ketobutyrate is abbreviated as α-KB.

Also included within the compounds of Formula I are a “glutarate compounds”, which to α-KG compounds, 2-HG compounds, and compounds having the following structural Formula III:

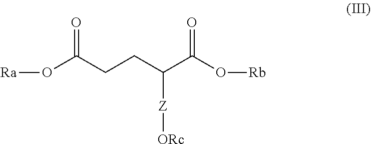

wherein
Ra and Rb are each independently a negative charge, H, Na, a straight or branched C1-C10 alkyl, or a straight or branched C1-C10 alkenyl, and
Rc is optionally present, and if present, Rc is H, a straight or branched C1-C10 alkyl, or a straight or branched C1-C10 alkenyl, and if absent, Z is a double bond,
and pharmaceutically acceptable solvates, salts, prodrugs, and metabolites thereof.

As used herein, a “C1-Cn alkyl” and a “C₁₋ₙ alkyl” refer to an alkyl having 1-n carbon atoms, where “n” is a positive integer. Similarly, a “C1-Cn alkenyl” and a “C₁₋ₙ alkenyl” refer to an alkenyl having 1-n carbon atoms, where “n” is a positive integer. The alkyls and alkenyls as set forth for Formula I, Formula II, and Formula III may be substituted or unsubstituted with one or more suitable functional groups that may increase or decrease, but not completely abrogate the ability of the compound to inhibit or reduce the activity NADH dehydrogenase.

As used herein, an “α-KG compound” refers to α-ketoglutarate (α-ketoglutarate), derivatives of α-ketoglutarate (e.g., the derivatives set forth in MacKenzie, et al. (2007) Mol Cell Biol 27(9):3282-3289)), analogues of α-ketoglutarate (e.g., phosphonate analogues (e.g., those recited in Bunik, et al. (2005) Biochemistry 44(31): 10552-61), esters of α-ketoglutarate (e.g., dimethyl α-ketoglutarate and octyl α-ketoglutarate), and various species specific analogues, e.g., human α-ketoglutarate, porcine α-ketoglutarate, murine α-ketoglutarate, bovine α-ketoglutarate, and the like. As used herein, the abbreviation “KG” may be used to refer to the term “ketoglutarate”, e.g., α-ketoglutarate is abbreviated as α-KG.

As used herein, a “2-HG compound” refers to 2-hydroxyglutaric acid, 2-hydroxypentanedioate, and compounds having 2-hydroxypentanedioate as part of its backbone structure and includes 1-alkyl-(S)-2-hydroxypentanedioate, 1-alkyl-(R)-2-hydroxypentanedioate, 1-alkenyl-(S)-2-hydroxypentanedioate, 1-alkenyl-(R)-2-hydroxypentanedioate, 5-alkyl-(S)-2-hydroxypentanedioate, 5-alkyl-(R)-2-hydroxypentanedioate, 5-alkenyl-(S)-2-hydroxypentanedioate, and 5-alkenyl-(R)-2-hydroxypentanedioate, wherein alkyl is a straight or branched C1-C10 alkyl and alkenyl is a straight or branched C1-C10 alkenyl. In some embodiments, the 2-HG compound is 1-octyl-(S)-2-hydroxypentanedioate, 1-octyl-(R)-2-hydroxypentanedioate, 5-octyl-(S)-2-hydroxypentanedioate, or 5-octyl-(R)-2-hydroxypentanedioate. In some embodiments, the 2-HG compound is disodium (S)-2-hydroxyglutarate (S-2HG, L-α-Hydroxyglutaric acid disodium salt). As used herein, the abbreviation “HG” may be used to refer to the term “hydroxypentanedioate”, e.g., 2-hydroxypentanedioate is abbreviated as 2-HG.

### Exemplary Treatment Methods

In some embodiments, the present invention is directed to a method of stimulating new hair growth in a subject in need thereof, which comprises administering to the subject a therapeutically effective amount of one or more compounds of Formula I, Formula II, and/or Formula III. In some embodiments, the one or more compounds are administered in the form of a pharmaceutical composition or formulation as described herein. In some embodiments, the one or more compounds, composition, or formulation, is administered to an area on the subject where new hair growth is desired.

In some embodiments, the present invention is directed to a method of stimulating new hair growth in a subject in need thereof, which comprises administering to the subject a pharmaceutical composition comprising a therapeutically effective amount of a compound of Formula I:

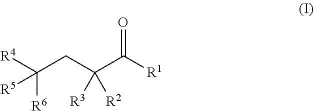

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl; R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo; R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or a salt thereof, and an excipient; wherein the pharmaceutical composition is administered to an area on the subject absent of hair to stimulate new hair growth.

In some embodiments of a compound of Formula I, R¹ is hydrogen, halogen, —CHO, —OR⁷, —NR⁸R⁹, —COOR⁷, —CONR⁸R⁹, or —SR¹⁰. In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is —OR⁷, wherein R⁷ is C₁₋₂₀ substituted or unsubstituted alkyl.

In some embodiments of a compound of Formula I, R² is hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl and R³ is hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl. In some embodiments, R² is hydrogen, halogen, —CN, —CHO, or —NR⁸R⁹, wherein R⁸ and R⁹ are each independently hydrogen or substituted or unsubstituted alkyl. In some embodiments, R³ is hydrogen, halogen, —CN, —CHO, or —NR⁸R⁹, wherein Re and R⁹ are each independently hydrogen or substituted or unsubstituted alkyl. In some embodiments, R² and R³, together with the atom to which they are bound, form an oxo.

In some embodiments of a compound of Formula I, R⁴ is hydrogen, —CHO, —OR⁷, —NR⁸R⁹, —COOR⁷, or —CONR⁸R⁹. In some embodiments, R⁵ is hydrogen, —CHO, —OR⁷, —NR⁸R⁹, —COOR⁷, or —CONR⁸R⁹. In some embodiments, R⁶ is hydrogen, —CHO, —OR⁷, —NR⁸R⁹, —COOR⁷, or —CONR⁸R⁹.

In some embodiments of a compound of Formula I, R⁷ is hydrogen or C1-20 substituted or unsubstituted alkyl. In some embodiments, Re is hydrogen or C₁₋₂₀ substituted or unsubstituted alkyl. In some embodiments, R⁹ is hydrogen or C₁₋₂₀ substituted or unsubstituted alkyl.

In some embodiments, the present is directed to a method of stimulating new hair growth in a subject in need thereof, which comprises administering to the subject a pharmaceutical composition comprising a therapeutically effective amount of a compound of Formula I:

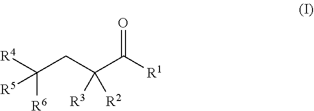

wherein
R¹ is —CHO, —OR⁷, —NR⁸R⁹, —COOR⁷, —CONR⁸R⁹, —SR¹⁰; R² and R³ are each independently hydrogen, halogen, —CN, —CHO, —OR⁷, —NR⁸R⁹, —SR¹⁰, or substituted or unsubstituted alkyl; or R² and R³, together with the atom to which they are bound, form an oxo; R⁴, R⁵, and R⁶ are each independently hydrogen, halogen, —OR⁷, —NR⁸R⁹, —COOR⁷, —CONR⁸R⁹, or substituted or unsubstituted alkyl; and R⁷, R⁸, R⁹, and R¹⁰ are each independently hydrogen, substituted or unsubstituted alkyl, or substituted or unsubstituted heteroalkyl; or a salt thereof; and an excipient; wherein the pharmaceutical composition is administered to an area on the subject absent of hair to stimulate new hair growth.

In some embodiments, the present invention is directed to a method of stimulating new hair growth in a subject in need thereof, which comprises administering to the subject a pharmaceutical composition comprising a therapeutically effective amount of a compound of Formula I:

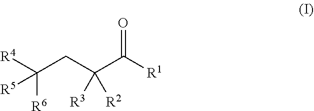

wherein
R¹ is —OR⁷ or —NR⁸R⁹; R² and R³ are each independently hydrogen, —CHO, —OR⁷, —NR⁸R⁹, or unsubstituted alkyl; or R² and R³, together with the atom to which they are bound, form an oxo; R⁴, R⁵, and R⁶ are each independently hydrogen, —OR⁷, —NR⁸R⁹, or unsubstituted alkyl; and R⁷, R⁸, R⁹, and R^{1′} are each independently hydrogen, or substituted or unsubstituted alkyl; or a salt thereof; and an excipient; wherein the pharmaceutical composition is administered to an area on the subject absent of hair to stimulate new hair growth.

In some embodiments, the present invention is directed to a method of stimulating new hair growth in a subject in need thereof, which comprises administering to the subject a pharmaceutical composition comprising a therapeutically effective amount of a compound of Formula I

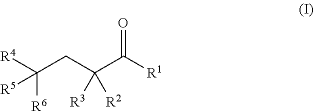

wherein
R¹ is —OR⁷; R² and R³, together with the atom to which they are bound, form an oxo; R⁴, R⁵, and R⁶ are each independently hydrogen or unsubstituted alkyl; and R⁷, R⁸, R⁹, and R¹⁰ are each independently hydrogen or unsubstituted alkyl; or a salt thereof; and an excipient; wherein the pharmaceutical composition is administered to an area on the subject absent of hair to stimulate new hair growth.

In some embodiments, the present invention is directed to a method of stimulating new hair growth in a subject in need thereof, which comprises administering to the subject a pharmaceutical composition comprising a therapeutically effective amount of a compound represented by the structure:

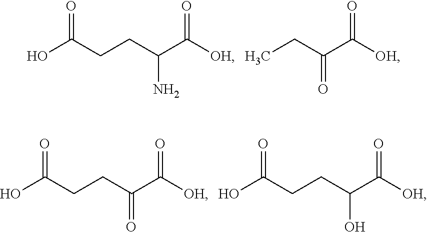

or a salt thereof; and an excipient; wherein the pharmaceutical composition is administered to an area on the subject absent of hair to stimulate new hair growth.

In some embodiments, the present invention is directed to a method of stimulating new hair growth in a subject in need thereof, which comprises administering to the subject a pharmaceutical composition comprising a therapeutically effective amount of a compound represented by the structure:

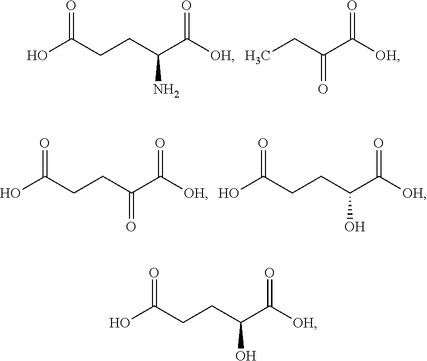

or a salt thereof; and an excipient; wherein the pharmaceutical composition is administered to an area on the subject absent of hair to stimulate new hair growth.

A “pharmaceutically acceptable solvate” refers to a solvate form of a specified compound that retains the biological effectiveness of the specified compound. Examples of solvates include compounds of the invention in combination with water, isopropanol, ethanol, methanol, dimethyl sulfoxide, ethyl acetate, acetic acid, ethanolamine, or acetone. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of compounds of Formula I, Formula II, and Formula III are within the scope of the invention. It will also be appreciated by those skilled in organic chemistry that many organic compounds can exist in more than one crystalline form. For example, crystalline form may vary from solvate to solvate. Thus, all crystalline forms of the compounds of Formula I, Formula II, Formula III, or the pharmaceutically acceptable solvates thereof are within the scope of the present invention.

A “pharmaceutically acceptable salt” refers to a salt form that is pharmacologically acceptable and substantially non-toxic to the subject being treated with the compound of the invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

A “pharmaceutically acceptable prodrug” is a compound that may be converted under physiological conditions or by solvolysis to the specified compound or to a pharmaceutically acceptable salt of such compound. “A pharmaceutically active metabolite” refers to a pharmacologically active product produced through metabolism in the body of a specified compound or salt thereof. Prodrugs and active metabolites of a compound may be identified using routine techniques known in the art. See, e.g., Bertolini, G. et al., (1997) J. Med. Chem. 40:2011-2016; Shan, D. et al., J. Pharm. Sci., 86(7):765-767; Bagshawe K., (1995) Drug Dev. Res. 34:220-230; Bodor, N., (1984) Advances in Drug Res. 13:224-331; Bundgaard, H., Design of Prodrugs (Elsevier Press, 1985) and Larsen, I. K., Design and Application of Prodrugs, Drug Design and Development (Krogsgaard-Larsen et al., eds., Harwood Academic Publishers, 1991).

As used herein, a “therapeutically effective amount” refers to an amount that may be used to treat, prevent, or inhibit a given disease or condition in a subject as compared to a control. For example, a therapeutically effective amount of one or more compounds of Formula I, Formula II, and/or Formula III is an amount that stimulates hair growth as compared to a negative control. Again, the skilled artisan will appreciate that certain factors may influence the amount required to effectively treat a subject, including the degree of the given disease or condition, previous treatments, the general health and age of the subject, and the like. Nevertheless, therapeutically effective amounts may be readily determined by methods in the art. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

### Additional Therapeutic Agents

A compound of Formula I, Formula II, and Formula III may be co-administered with an additional agent. The additional agent may comprise one or more growth factors. In some embodiments, the growth factor comprises TGF-β2, IGF-1, KGF or HGF.

As used herein, “co-administered” refers to the administration of at least two different agents (a first and second agent, e.g., a compound of Formula I and an additional agent; or a compound of Formula II and a compound of Formula III) to a subject. In some embodiments, the co-administration is concurrent. In embodiments involving concurrent co-administration, the agents may be administered as a single composition, e.g., an admixture, or as two separate compositions. In some embodiments, a compound of Formula I, Formula II, and Formula III is administered before and/or after the administration of the second agent, e.g., the additional agent. Where the co-administration is sequential, the administration of the first and second agents may be separated by a period of time, e.g., minutes, hours, or days. Those of skill in the art understand that the formulations and/or routes of administration of the various agents or therapies used may vary. The appropriate dosage for co-administration can be readily determined by one skilled in the art. In some embodiments, when two or more agents are co-administered, the respective agents are administered at lower dosages than appropriate for their administration alone.

Growth factors are substances capable of stimulating cellular growth, proliferation, healing, and cellular differentiation. There are several families and types of growth factors, and are involved in wide range of processes. Growth factors typically act as signaling molecules between cells. Different growth factor families including TGF-β2, IGF-1, KGF and HGF have been shown to be crucial for the regulation of the hair cycle and hair growth.

Transforming growth factor-beta (TGF-β) is a multifunctional cytokine belonging to the transforming growth factor superfamily that includes three different isoforms (TGF-β1 to 3). Transforming growth factor-beta 2, or TGF-β2, is a secreted protein known as a cytokine that performs many cellular functions and has vital role during embryonic development. It is also known as Glioblastoma-derived T-cell suppressor factor, G-TSF, BSC-1 cell growth inhibitor, Polyergin, and Cetermin. TGF-β2 is localized around hair follicles, implicating their role during hair morphogenesis. In some embodiments, TGF-β2 is administered to a subject in combination with an α-ketobutyrate compound and/or a glutarate compound described herein.

Insulin-like growth factor-1 (IGF-1), also called somatomedin C, is a hormone similar in molecular structure to insulin. IGF-1 stimulates replication of mesenchymal and epithelial cells and influences epithelial elements of the hair organ and stimulates hair follicle growth in dose-dependent manner. It is also able to suppress hair follicle entry into a catagen-like state. In some embodiments, IGF-1 is administered to a subject in combination with an α-ketobutyrate compound and/or a glutarate compound described herein.

Keratinocyte growth factor (KGF), also known as FGF-7, belongs to the Fibroblast growth factor (FGF) family and is synthesized by stromal fibroblasts. KGF is upregulated during wound healing and accelerates reepithelization and dermal regeneration. KGF also increases the number of hair follicles and proliferating cells in a dose-dependent manner. In some embodiments, KGF (FGF-7) is administered to a subject in combination with an α-ketobutyrate compound and/or a glutarate compound described herein.

Hepatocyte growth factor (HGF) is a paracrine cellular growth, motility and morphogenic factor. HGF promotes hair growth in a dose dependent manner. In some embodiments, HGF is administered to a subject in combination with an α-ketobutyrate compound and/or a glutarate compound described herein.

### Pharmaceutical Compositions and Formulations

The one or more compounds of Formula I, Formula II, and/or Formula III (e.g., two different compounds of Formula I; a compound of Formula II and a compound of Formula III such as one or more α-KB compounds and/or one or more glutarate compounds; etc.) to be administered to a subject may be administered as a pharmaceutical composition or formulation. The pharmaceutical compositions and formulations may further include an additional agent as described above.

In some embodiments, a therapeutically effective amount of one or more α-KB compounds and/or one or more glutarate compounds is administered as a daily dose of about 0.01-2, about 0.25-2, about 0.5-2, about 1-2, or about 2 grams per kilogram weight of the subject per day. In some embodiments, a therapeutically effective amount of one or more α-KB compounds and/or one or more glutarate compounds is administered as a daily dose of about 0.1-1, about 0.25-1, about 0.5-1, or about 1 gram per kilogram weight of the subject per day. In some embodiments, one or more α-KB compounds and/or one or more glutarate compounds is administered as a daily dose of about 0.01-1.0, about 0.01-0.5, or about 0.1-0.2 grams per kilogram weight of the subject per day. The skilled artisan will appreciate that certain factors may influence the dosage required to effectively treat a subject, including the severity of the disease or disorder, previous treatments, the general health and/or age of the subject, and other diseases present.

The therapeutically effective amount may be administered as a single dose or as multiple doses (e.g., 2, 3, 4, 5, 6, 7, 8, 9, 10, or more doses) over a period of time. For example, a subject may be treated with one or more α-KB compounds and/or one or more glutarate compounds at least once. Alternatively, the subject may be treated with one or more α-KB compounds and/or one or more glutarate compounds from about one time per week to about once daily for a given treatment period. The length of the treatment period will depend on a variety of factors such as the severity of the disease or disorder, the concentration and activity of the one or more compounds of the present invention, or a combination thereof. It will also be appreciated that the effective dosage of the one or more compounds used for treatment may increase or decrease over the course of a particular treatment.

The one or more compounds of Formula I, Formula II, and/or Formula III (e.g., one or more α-KB compounds and/or one or more glutarate compounds) to be administered to a subject may be provided as a pharmaceutical formulation. Pharmaceutical formulations may be prepared in a unit-dosage form appropriate for the desired mode of administration. The pharmaceutical formulations of the present invention may be administered by any suitable route including oral, rectal, nasal, topical (including buccal and sublingual), vaginal, and parenteral (including subcutaneous, intramuscular, intravenous, and intradermal). It will be appreciated that the route of administration may vary with the condition and age of the recipient, the nature of the condition to be treated, and the given compound(s) of the present invention. In some embodiments, the route of administration is oral. In some embodiments, the one or more compounds of Formula I, Formula II, and/or Formula III are provided in the form of a foodstuff.

It will be appreciated that the actual dosages of the one or more compounds of Formula I, Formula II, and/or Formula III (e.g., one or more α-KB compounds and/or one or more glutarate compounds) used in the pharmaceutical formulations will vary according to the specific compound(s) being used, the particular composition formulated, the mode of administration, and the particular site, subject, and disease being treated. Optimal dosages for a given set of conditions may be ascertained by those skilled in the art using dosage determination tests in view of the experimental data for a given compound. Administration of prodrugs may be dosed at weight levels that are chemically equivalent to the weight levels of the fully active forms.

In some embodiments, pharmaceutical compositions of the present invention comprise a therapeutically effective amount of one or more compounds of Formula I, Formula II, and/or Formula III, and a pharmaceutically acceptable carrier or diluent. As used herein “pharmaceutically acceptable carrier” include solvents, dispersion media, coatings, antibacterial, and antifungal agents, isotonic and absorption delaying agents, stabilizers, diluents, suspending agents, thickening agents, excipients, and the like, compatible with pharmaceutical administration. Pharmaceutical compositions are optionally manufactured using methods in the art, e.g., by mixing, dissolving, granulating, dragee-making, levigating, emulsifying, encapsulating, entrapping or compression processes.

In some embodiments, the pharmaceutical compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases, and buffers may be included in an amount required to maintain pH of the composition in an acceptable range.

In some embodiments, pharmaceutical compositions of the present invention may also include one or more salts in an amount to bring osmolality of the composition into a desired range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

As used herein, a “pharmaceutical combination” refers to a product that results from the mixing or combining of more than one active ingredient and includes both fixed and non-fixed combinations of the active ingredients. The term “fixed combination” means that the active ingredients (e.g., a compound of Formula I, Formula II, or Formula III and an additional agent) are administered to a patient simultaneously in the form of a single entity or dosage. The term “non-fixed combination” means that the active ingredients are administered to a subject as separate entities either simultaneously, concurrently, or sequentially with no specific intervening time limits, wherein such administration provides effective levels of the two compounds in the body of the patient. The latter also applies to cocktail therapy, e.g., the administration of three or more active ingredients.

The pharmaceutical compositions may be formulated into any suitable dosage form, including aqueous oral dispersions, liquids, gels, syrups, elixirs, slurries, suspensions, and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (e.g., for IV administration).

In some embodiments, using coating procedures known in the art such as those described in Remington's Pharmaceutical Sciences, 20th Edition (2000), a film coating is provided around the pharmaceutical compositions. In some embodiments, the pharmaceutical compositions are formulated into particles (e.g., for administration by capsule) and some or all the particles are coated. In some embodiments, the pharmaceutical compositions are formulated into particles (e.g., for administration by capsule) and some or all the particles are microencapsulated. In some embodiments, the compositions are formulated into particles (e.g., for administration by capsule) and some or all the particles are not microencapsulated and are uncoated.

The pharmaceutical solid dosage forms include one or more compounds of Formula I, Formula II, and/or Formula III, and may optionally include one or more pharmaceutically acceptable additives such as pharmaceutically acceptable carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, and preservatives.

In some embodiments, the pharmaceutical compositions may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

An “antifoaming agent” reduces foaming during processing which can result in coagulation of aqueous dispersions, bubbles in the finished film, or generally impair processing. Exemplary anti-foaming agents include silicon emulsions or sorbitan sesquoleate.

Examples of“antioxidants” include butylated hydroxytoluene (BHT), sodium ascorbate, ascorbic acid, sodium metabisulfite and tocopherol. In some embodiments, antioxidants enhance chemical stability.

The pharmaceutical formulations may include one or more antioxidants, metal chelating agents, thiol containing compounds, and/or stabilizing agents. Examples of such stabilizing agents, include: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

A “binders” imparts cohesive qualities and include alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

As used herein, “pharmaceutically acceptable carriers” include carriers and excipients used in pharmaceutics and should be selected on the basis of compatibility with the active ingredients, e.g., one or more compounds of Formula I, Formula II, and/or Formula III, and the release profile properties of the desired dosage form. Exemplary carriers include, binders, suspending agents, disintegration agents, filling agents, surfactants, solubilizers, stabilizers, lubricants, wetting agents, diluents, and the like. Exemplary carrier materials include acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

As used herein, “dispersing agents” and/or “viscosity modulating agents” include materials that control the diffusion and homogeneity of a drug through liquid media or a granulation method or blend method. In some instances, these agents also facilitate the effectiveness of a coating or eroding matrix. Exemplary diffusion facilitators/dispersing agents include hydrophilic polymers, electrolytes, Tween® 60 or 80, PEG, polyvinylpyrrolidone (PVP; commercially known as Plasdone®), and the carbohydrate-based dispersing agents such as hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as gum tragacanth and gum acacia, guar gum, xanthans, including xanthan gum, sugars, cellulosics, such as sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol, and isopropyl myristate.

The pharmaceutical compositions may also include one or more erosion facilitators and/or one or more diffusion facilitators.

A “diluent” is chemical compound that is used to dilute the concentration of a given compound in a composition. Diluents can also be used to stabilize compounds by providing a more stable environment. Salts dissolved in buffered solutions (which also can provide pH control or maintenance), such as phosphate buffered saline solutions, may be used as diluents. In some instances, diluents increase bulk of the composition to facilitate compression or create sufficient bulk for homogenous blend for capsule filling. Such bulk increasing diluents include lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

A “disintegration agent”, also referred to as a “disintegrant”, facilitates the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

As used herein, “drug absorption” or “absorption” refers to the process of movement of drug from site of administration of a drug across a barrier into the site of action, e.g., a drug moving from the gastrointestinal tract into the portal vein or lymphatic system, or a drug passing from the surface of the skin to, e.g., the hair follicle.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

As used herein, “erosion facilitators” include materials that control the erosion of a particular material in gastrointestinal fluid. Erosion facilitators are generally known to those of ordinary skill in the art. Exemplary erosion facilitators include hydrophilic polymers, electrolytes, proteins, peptides, and amino acids.

As used herein, “filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

As used herein, “flavoring agents” and/or “sweeteners” useful in the formulations described herein, include acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

A “lubricant” also referred to as a “glidant” is a compound that prevents, reduces, or inhibits adhesion or friction of materials. Exemplary lubricants include stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

The term “pharmacodynamics” refers to the factors which determine the biologic response observed relative to the concentration of drug at a site of action.

The term “pharmacokinetics” refers to the factors which determine the attainment and maintenance of the appropriate concentration of drug at a site of action.

A “plasticizer” is a compound used to soften the microencapsulation material or film coatings to make them less brittle. Suitable plasticizers include polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

Exemplary “solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide, and the like.

As used herein, “stabilizers” include compounds such as any antioxidation agents, buffers, acids, preservatives, and the like.

As used herein, “steady state” refers to the state in which the amount of drug administered is equal to the amount of drug eliminated within one dosing interval resulting in a plateau or constant plasma drug exposure.

Exemplary “suspending agents” include polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as gum tragacanth and gum acacia, guar gum, xanthans, including xanthan gum, sugars, cellulosics, such as sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

Exemplary “surfactants” include sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

Exemplary “viscosity enhancing agents” include methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Exemplary “wetting agents” include oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

### Dosage Forms

The pharmaceutical formulations of the present invention may be administered by any suitable route including oral, rectal, nasal, topical (including buccal and sublingual), vaginal, and parenteral (including subcutaneous, intramuscular, intravenous, and intradermal). Furthermore, the pharmaceutical compositions according to the present invention can be formulated into any suitable dosage form, including aqueous oral dispersions, liquids, gels, syrups, elixirs, slurries, suspensions, and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Topical Formulations

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In some embodiments, the topical formulation is presented in a liquid dosage form, conveniently packaged in single or multiple units, which may comprise one or more pharmaceutically acceptable excipients. In some embodiments, the liquid dosage form is a aqueous-based, alcohol-based or hydro-alcohol based composition wherein the said alcohol refers to class of lower alcohols. In some embodiments, the topical formulation includes suitable excipients which increase the viscosity of the formulation to provide range of viscous liquid to semisolid consistency based formulation.

In some embodiments, the topical formulation is aqueous based with minimum amount of alcohols (e.g., lower alcohols) or totally devoid of alcohols (e.g., lower alcohols). In some embodiments, the topical formulation is an alcohol or hydro-alcohol based composition which comprise pharmaceutically suitable amount of alcohol ranging from 0% to 10% with one or more pharmaceutically acceptable excipients.

In some embodiments, the topical formulation is an aqueous based composition which comprises the compounds described herein with one or more of pharmaceutically acceptable excipients wherein the said composition i) is totally devoid of excipients like propylene glycol and/or lower alcohols, or, ii) comprise less than 10% of excipients like propylene glycol and/or lower alcohols.

One or more pharmaceutically acceptable carriers or excipients are used for formulating the topical formulation according to the present invention.

Suitable carriers and excipients include one or more of surfactants/wetting agents, acidifying agents, solubilizers, penetration enhancers, preservatives, humectants, moisturizers, anti-oxidants, de-tackifying agents, conditioning agents, proteins, fragrances, and mixtures thereof.

Surfactants/wetting agents include anionic, cationic, nonionic, zwitterionic, amphoteric and ampholytic surfactants, as well as mixtures thereof. Examples of suitable surfactants and wetting agents, include polyethoxylated fatty acids, fatty acid diesters, polyethylene glycol glycerol fatty acid esters, alcohol-oil transesterification products, polyglycerized fatty acids, sterol and sterol derivatives, polyethylene glycol sorbitan fatty acid esters/Polysorbates; polyethylene glycol alkyl ethers, sugar esters, polyethylene glycol alkyl phenols, polyoxyethylene-polyoxypropylene block copolymers, sorbitan fatty acid esters and lower alcohol fatty acid esters; polyoxyethylene (POE) fatty acid esters, such as Myrj®; polyoxyethylene alkylyl ethers, such as poly oxyethylene cetyl ether, polyoxyethylene palmityl ether, polyethylene oxide hexadecyl ether, polyethylene glycol cetyl ether, Brij; Sodium dodecyl sulfate (sodium lauryl sulfate), Lauryl dimethyl amine oxide, Docusate sodium, Cetyl trimethyl ammonium bromide (CTAB); Octoxynol; N, N-dimethyldodecylamine-N-oxide; Hexadecyltrimethylammonium bromide; Polyoxyl 10 lauryl ether; Bile salts (sodium deoxycholate, sodium cholate); Methicones; Polyoxyl castor oil; Nonylphenol ethoxylated Cyclodextrins; Lecithins; Methylbenzethonium chloride; Glycol esters of fatty acids, Carboxylic amides, Monoalkanolamine condensates, Polyoxyethylene fatty acid amides, Quaternary ammonium salts, Polyoxyethylene alkyl and alicyclic amines or mixtures thereof. In some embodiments, the topical formulation comprises one or more surfactants/wetting agents in an amount ranging from about 1% w/v to about 10% w/v.

Suitable solubilizers comprises one or more of glycerols; glycols such as polyethylene glycols of various grades; aliphatic alcohols/aromatic alcohols; Polyoxyl n castor oil (synonyms—ethoxylated castor oil, polyethylene glycol castor oil and wherein “n” is the number of oxyethylene units in the compound); Polyoxyl n hydrogenated castor oil or mixtures thereof. In some embodiments, the topical formulation comprises one or more solubilizers in an amount ranging from about 1% w/v to about 50% w/v.

Suitable penetration enhancers comprises one or more of glycol ether solvents such as Ethylene glycol monomethyl ether, Ethylene glycol monoethyl ether, Ethylene glycol monopropyl ether, Ethylene glycol monoisopropyl ether, Ethylene glycol monobutyl ether, Ethylene glycol monophenyl ether, Ethylene glycol monobenzyl ether, Diethylene glycol monomethyl ether, Diethylene glycol monoethyl ether, Diethylene glycol mono-n-butyl ether; dialkyl ethers and dialkyl ether esters such as ethylene glycol dimethyl ether, ethylene glycol diethyl ether, ethylene glycol dibutyl ether, and ethylene glycol methyl ether acetate, ethylene glycol monoethyl ether acetate, ethylene glycol monobutyl ether acetate or mixtures thereof. In some embodiments, the topical formulation comprises one or more penetration enhancers in an amount ranging from about 1% w/v to about 20% w/v. Suitable acidifying agents include one or more of acetic acid, hydrochloric acid, salicylic acid, boric acid, sulfuric acid, lactic acid, and citric acid or mixtures thereof. In some embodiments, the topical formulation comprises one or more acidifying agents in an amount ranging from about 0.5% w/v to about 10% w/v.

Suitable preservatives include one or more of aliphatic or aromatic alcohols; glycols; parahydroxybenzoic acid derivatives (e.g., parabens); Vitamin E or its derivatives which may include ethyl alcohol, benzyl alcohol, propylene glycol, glycerin, benzoic acid/sodium benzoate, sorbic acid, methylparaben, propylparaben, benzalkonium chloride or mixtures thereof. In some embodiments, the topical formulation comprises one or more preservatives in an amount ranging from about 0.1% w/v to about 10% w/v.

Suitable de-tackifying agents include one or more of silanes; methicones; alkyl/aryl lactates or mixtures thereof. In some embodiments, the topical formulation comprises one or more de-tackifying agents in an amount ranging from 0.1% w/v. to about 15% w/v.

In some embodiments, the topical formulation comprises one or more surfactants, one or more solubilisers, one or more penetration enhancers, one or more acidifying agents, one or more preservatives and one or more detackifying agents.

In some embodiments, the topical formulation comprises at least one additional ingredient such as finasteride, dutasteride, ketoconazole, and in case of female androgenic alopecia, other drugs that are used include spironolactone, alfatradiol, or flutamide, vitamins (water soluble or fat soluble or both), biotin, D-panthenol, niacinamide; herbal extracts and dietary supplements, e.g., saw palmetto (Serenoa repens), stinging nettle (Urtica dioica), turmeric (Curcubita pepo), and Pygeum africanum. Other herbs include black cohosh (Actaea racemosa), dong quai (Angelica sinensis), false unicorn (Chamaelirium luteum), chasteberry (Vitex agnus-castus), red clover (Trifolium pratense), L-arginine, Boswellia serrata, L-Carnitine, curcumin, ginger, grape seed extract, Grateloupia elliptica, green tea, lycopene, pumpkin seed oil (Curcurbitae pepo), and resveratrol.

In some embodiments, the one or more additional ingredients may be presented in combination with the topical formulation as a fixed and single presentation or as separate kit presentation either solely in the form of topical route or in the form of combination of topical route and other than topical route (which may include oral route) presentations.

In some embodiments, the topical formulation is a liquid composition that is administered using a spray device lacking a chemical propellant, a dropper, or otherwise mechanically spread on the scalp or skin without the use of a propellant. Such a liquid composition comprises the compounds disclosed herein or a pharmaceutically acceptable salt thereof, aluminum starch octenyl succinate, and a pharmaceutically acceptable solvent. The solvent is one readily apparent to one in the field, and comprises one or more of the carrier materials and the solvents discussed under the aerosol composition (nonfoaming). In some embodiments, the compositions described herein are formulated for application as a solution spray. The composition described herein is applied directly to the hair or scalp, by using a container fitted with a pump to dispense a liquid composition (e.g., an atomizer), or by a pump aerosol container which utilizes compressed air as the propellant.

In some embodiments, the liquid composition contains aluminum starch octenyl succinate at the weight percentages discussed concerning the aerosol composition (non-foaming). Additional adjuvants may be used in the formulation according to the disclosure herein of suitable adjuvants. A suitable solvent is water adjusted to a pH to allow for dissolution of all the composition components.

### Oral Formulations

Pharmaceutical compositions formulated for oral use are obtained by mixing one or more solid excipient with one or more of compounds of Formula I, Formula II, and/or Formula III, optionally grinding the resulting mixture, and processing the mixture of granules, after adding suitable auxiliaries, if desired, to obtain tablets or dragee cores. Examples of suitable excipients include fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores may be provided with suitable coatings. For this purpose, concentrated sugar solutions are used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In some embodiments, the oral formulations according to the present invention include solid dosage forms in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical formulation is in the form of a powder. In some embodiments, the pharmaceutical formulation is in the form of a tablet, including a fast-melt tablet. In some embodiments, the oral formulations described herein are administered as a single capsule or in multiple capsule dosage form. In some embodiments, the pharmaceutical formulation is administered in two, or three, or four, capsules or tablets.

In some embodiments, the pharmaceutical solid dosage forms include a composition described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some embodiments, the solid dosage forms include a coating such as those described in Remington's Pharmaceutical Sciences, 20th Edition (2000).

Suitable carriers for use in the solid dosage forms include acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol, and the like.

Suitable filling agents for use in the solid dosage forms include lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Binders impart cohesiveness to solid oral dosage form formulations: for powder filled capsule formulation, they aid in plug formation that are filled into soft or hard shell capsules and for tablet formulation, they ensure the tablet remaining intact after compression and help assure blend uniformity prior to a compression or fill step. Materials suitable for use as binders in the solid dosage forms described herein include carboxymethylcellulose, methylcellulose (e.g., Methocel®), hydroxypropylmethylcellulose (e.g., Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Suitable lubricants or glidants for use in the solid dosage forms include stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms include sugars (including lactose, sucrose, and dextrose), polysaccharides (including dextrates and maltodextrin), polyols (including mannitol, xylitol, and sorbitol), cyclodextrins, and the like.

Suitable wetting agents for use in the solid dosage forms include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS, and the like.

Suitable surfactants for use in the solid dosage forms include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms include polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as gum tragacanth and gum acacia, guar gum, xanthans, including xanthan gum, sugars, cellulosics, such as sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

Suitable antioxidants for use in the solid dosage forms include, for example, e.g., butylated hydroxytoluene (BHT), sodium ascorbate, and tocopherol.

Liquid formulation dosage forms for oral administration include aqueous suspensions selected from the group including pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystalline inhibitor.

In some embodiments, the aqueous suspensions and dispersions described herein remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In some embodiments, an aqueous suspension is re-suspended into a homogenous suspension by physical agitation lasting less than 1 minute. In another aspect, an aqueous suspension is re-suspended into a homogenous suspension by physical agitation lasting less than 45 seconds. In some embodiments, an aqueous suspension is re-suspended into a homogenous suspension by physical agitation lasting less than 30 seconds. In some embodiments, no agitation is necessary to maintain a homogeneous aqueous dispersion.

In some embodiments, dosage forms include microencapsulated formulations. In some embodiments, one or more other compatible materials are present in the microencapsulation material. Exemplary materials include pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including compounds described herein, include hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Plasticizers include polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In some embodiments, the microencapsulating material useful for delaying the release of the pharmaceutical compositions is from the USP or the National Formulary (NF). In some embodiments, the microencapsulation material is Klucel. In some embodiments, the microencapsulation material is methocel.

Microencapsulated compositions are formulated by methods known by one of ordinary skill in the art. Such known methods include spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media may also used. Furthermore, other methods such as roller compaction, extrusion/spheronization, coacervation, or nanoparticle coating may be used.

### Aerosol, Non-Foaming Compositions

In some embodiments, the pharmaceutical formulation is an aerosol composition comprising a dry shampoo and a propellant, wherein the composition is not formulated for application as a foam or mousse. By “dry shampoo” is meant a formulation comprising a carrier material that is a volatile liquid and therefore evaporates and a powder that remains, e.g., a starch or modified starch, such as aluminum starch octenyl succinate. In some embodiments, the dry shampoo comprises one or more compounds of Formula I, Formula II, and/or Formula III, a carrier material. Examples of suitable carrier materials that are volatile liquids are lower alcohols including without limitation ethanol or isopropanol, a volatile silicone compound such as polydimethylsiloxanes (e.g., having a viscosity less than about 5 cSt at 25° C.), cyclomethicone, cyclohexane siloxane, decamethyltetrasiloxane, octamethyltrisiloxane, decamethylpentasiloxane, decamethylcyclopentasiloxane, octamethylcyclotetrasiloxane, trimethylsilylamodimethicone, phenyl trimethicone, hexamethyidisiloxane, and dimethylsiloxane/methylalkylsiloxane, and combinations thereof. Other carrier materials known to those skilled in the art may also be used. The total percentage weight of the carrier material in the aerosol composition may be between about 0.1% and about 50%, between about 0.1% and about 40%, between about 1% and about 35%, between about 5% and about 50%, between about 10% and about 40%, between about 15% and about 40%, of the total weight of the aerosol dry shampoo composition (a combination of the dry shampoo composition and the propellant). In some embodiments, the composition is substantially free of water.

In some embodiments, the dry shampoo comprises a solvent, which may or may not be volatile. The solvent can be an alcohol, such as a polyhydric alcohol. Examples of polyhydric alcohols include 1,3-butylene glycol, propylene glycol, glycol 200 (PEG 200), polyethylene glycol400 (PEG 400), hexylene glycol and dipropylene glycol, and glycerol. The polyhydric alcohol can have a concentration of 10% or less by weight, or 5% or less by weight, or between 10% to 1% by weight. In some embodiments, the solvent can comprise benzyl alcohol. In some embodiments, the solvent comprises propylene glycol and/or water. Low levels of water (such as 1-5% or less than 10%) are preferred in the non-foaming aerosol compositions of the invention. If the solvent is non-volatile, low levels of less than 20%, less than 10%, less than 5% or less than 1% of the total weight of the aerosol dry shampoo composition (a combination of the dry shampoo composition and the propellant) is preferred.

In some embodiments, the dry shampoo comprises a starch or modified starch. In some embodiments, the starch or modified starch acts as a sebum absorber. Examples of suitable starch materials include cornstarch, potato starch, tapioca starch, rice starch, wheat starch, and cassaya starch. A starch material may be modified or unmodified. A modified starch material is a starch which has been derivatized or altered by processes known to those of ordinary skill in the art, such as esterification, etherification, oxidation, acid hydrolysis, crosslinking, or enzyme conversion. Examples of suitable modified starch materials include aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch, and sodium starch glycolate. In some embodiments, the dry shampoo comprises aluminum starch octenyl succinate and tapioca starch.

In some embodiments, the starch material may be present in the dry shampoo at a concentration of 1% to 70% by weight, 1% to 60% by weight, 1% to 50% by weight, 1% to 40% by weight, 1% to 30% by weight, 1% to 20% by weight, 1% to 15% by weight, 1% to 10% by weight, 5% to 50% by weight, 5% to 40% by weight, 5% to 30% by weight, 5% to 20% by weight, 5% to 10% by weight, 5% to 15% by weight, 10% to 60% by weight, 10% to 50% by weight, 10% to 40% by weight, 10% to 30% by weight, 10% to 20% by weight, 10% to 15% by weight, or 20% to 60% by weight as measured based on the total weight of the dry shampoo.

In some embodiments, the aerosol composition comprises a dry shampoo having as a starch or modified starch aluminum starch octenylsuccinate at a concentration in the dry shampoo of 2% to 20% by weight, 2% to 10% by weight, 10% to 20% by weight, 11% to 20% by weight, 10% to 15% by weight, 14 to 16% by weight, 2% to 10% by weight, 2 to 3% by weight, 3% to 4% by weight, 5% to 6% by weight, 7% to 8% by weight, 9% to 10% by weight, 11% to 12% by weight, 13% to 14% by weight, 13% to 15% by weight, 15% to 16% by weight, 17% to 18% by weight, 13% to 16% by weight, 14% to 16% by weight, 19% to 20% by weight, 2 to 8% by weight, 2 to 5% by weight, 4 to 8% by weight, or 5 to 10% by weight, or at a concentration of about 5%, 6%, 7%, 8%, 9%, 10%, 11%, 12%, 13%, 14%, 15%, 16%, 17%, 18%, 19%, or 20% by weight.

In some embodiments, the dry shampoo further comprises oil-absorbing powder that may be in addition to the starch or modified starch. Examples of oil-absorbing powders include cellulose, chalk, talc, fuller's earth, etc. In some embodiments, the dry shampoo includes a sebum absorber such as a clay material, e.g., stearalkonium hectorite. In some embodiments, the sebum absorber is at least one modified clay material selected from the group consisting of stearalkonium hectorite, stearalkonium bentonite, quaternium-18 bentonite, and quaternium-18 hectorite. In some embodiments, the dry shampoo includes silica, which may function as an oil-absorbing compound and/or a suspending agent. In some embodiments, the dry shampoo is substantially free of silica and silica-containing components.

In some embodiments, the aerosol compositions include a propellant. Examples of suitable propellants include butane, isobutane, propane, A-46 (isobutane and propane), liquefied petroleum gas (e.g., propane), dimethyl ether, methyl ethyl ether, trichlorofluoromethane, dichlorodifluoromethane, dichlorotetrafluorothane, monochlorodifluoromethane, trichlorotrifluoroethane propane, carbon dioxide, nitrous oxide, 1,1,1,2-tetrafluoroethane, 1,1,2,3,3,3-heptafluoropropane, or combinations thereof. In some embodiments, the propellant is isobutane. A propellant may condense to a liquid state in an aerosol container at ambient temperatures. In some embodiments, the propellant may have a lower specific gravity as compared to the rest of the composition, thus facilitating propelling the composition from a container (e.g., through a dip tube) as compared to expelling the propellant.

In some embodiments, the propellant is present at a concentration of 25% to 90% by weight, 25% to 80% by weight, 25% to 70% by weight, 25% to 60% by weight, 25% to 50% by weight, 25% to 40% by weight, 25% to 30% by weight, 30% to 90% by weight, 30% to 80% by weight, 30% to 70% by weight, 30% to 60% by weight, 30% to 50% by weight, 30% to 40% by weight, 40% to 90% by weight, 40% to 80% by weight, 40% to 70% by weight, 40% to 60% by weight, 40% to 50% by weight, 50% to 90% by weight, 50% to 80% by weight, 50% to 70% by weight, or 50% to 60% by weight of the total composition (dry shampoo and propellant).

In some embodiments, the pharmaceutical composition is formulated into a foam composition. In some embodiments, the foam compositions comprise one or more compounds of Formula I, Formula II, and/or Formula III, a starch or a modified starch such as aluminum starch octenylsuccinate, a gelling agent, and a propellant. In some embodiments, the foam composition is breakable foam that breaks upon application of shear pressure. In some embodiments, the propellant is any of the propellants discussed concerning the aerosol composition (non-foaming). In some embodiments, the gelling agent is any of those known to one of ordinary skill in the art, such as methylcellulose, poloxamers, bentonite, gelatin, sodium carboxymethyl cellulose, carbomers, tragacanth, and alginic acid.

In some embodiments, the pharmaceutical composition is formulated into a gel composition. In some embodiments, the gel compositions comprise one or more compounds of Formula I, Formula II, and/or Formula III, a starch or a modified starch such as aluminum starch octenylsuccinate, a gelling agent, and a solvent. In some embodiments, the gelling agent is any of those known in the art including the gelling agents discussed herein concerning the foam composition. In some embodiments, the solvent is any suitable for dissolving or suspending the compounds disclosed herein and the gelling agent.

Pharmaceutical formulations of this invention comprise a therapeutically effective amount of one or more compounds of the present invention, and an inert, pharmaceutically acceptable carrier or diluent. As used herein the language “pharmaceutically acceptable carrier” is intended to include any and all solvents, dispersion media, coatings, antibacterial, and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid, and the like. Exemplary of liquid carriers are syrup, peanut oil, olive oil, water, and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate, and the like. The use of such media and agents for pharmaceutically active substances is known in the art.

Toxicity and therapeutic efficacy of the one or more α-KB compounds and/or one or more glutarate compounds can be determined by standard pharmaceutical procedures in cell cultures or experimental animals, e.g., for determining the LDso (the dose lethal to 50% of the population) and the EDso (the dose therapeutically effective in 50% of the population). The dose ratio between toxic and therapeutic effects is the therapeutic index and it can be expressed as the ratio LDso/EDso. Compounds exhibiting large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the EDsa with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the ICso (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Kits and Article of Manufacture

In some embodiments, the present invention is directed to kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture contain packaging materials. Examples of pharmaceutical packaging materials include blister packs, bottles, tubes, bags, containers, bottles, and any packaging material suitable for a selected formulation and intended mode of administration and treatment.

For example, the container(s) include one or more compounds of Formula I, Formula II, and/or Formula III such as α-KG, α-KB and/or 2-HB, optionally in a composition or in combination with one or more growth factors disclosed herein. Such kits optionally include an identifying description or label or instructions relating to its use in the methods described herein.

In some embodiments, the kits include labels listing contents and/or instructions for use, and package inserts with instructions for use. In some embodiments, the kits include a set of instructions.

In some embodiments, a label is on or associated with at least one of the containers. In some embodiments, a label is on a container when letters, numbers or other characters forming the label are attached, molded or etched into the container itself, a label is associated with a container when it is present within a receptacle or carrier that also holds the container, e.g., as a package insert. In some embodiments, a label is used to indicate that the contents are to be used for a specific therapeutic application. In some embodiments, the label may include directions for use of the contents, such as in the methods described herein.

In some embodiments, the pharmaceutical compositions are presented in a pack or dispenser device which contains one or more unit dosage forms as described herein. The pack, for example, contains metal or plastic foil, such as a blister pack. In some embodiments, the pack or dispenser device is accompanied by instructions for administration. In some embodiments, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are also prepared, placed in an appropriate container, and labeled for treatment of an indicated condition.

### EXAMPLES

The following examples are intended to illustrate but not to limit the invention.

### Example 1

To investigate the effects α-KB, 2-HB, and α-KG have on stimulating hair growth, shaved young (6-week old) C57/B6 mice were topically treated and monitored for pigmentation as well as hair changes over the course of 3 weeks. Compounds were dissolved in Transdermal Gel at 16 mM and applied on mice dorsal skin every other day. To show the pigmentation progression, the appearance of skin pigmentation, signaling the initiation of anagen, was assigned an arbitrary value based on skin darkening. No hair growth (and no pigmentation) was assigned a value of 0 and darker skin/visible hair growth was assigned with higher numbers. Photos were taken weekly to document the pigmentation/hair changes.

Additionally, α-KG was administrated in drinking water to 6-week old C57/B6 mice for 10 weeks. α-KG was dissolved at 16 mM and water was changed every other day to ensure the integrity of the compound. Oral administration of α-KB was tested in 101-week old C57/B6 mice and lasted for 30 weeks. The concentration of α-KB was 8 mM and water was changed weekly. Pigmentation/hair changes were also monitored by assigning arbitrary values and photos.

### Treatment of α-KB Maintains Hair Pigmentation and Density in Old Mice

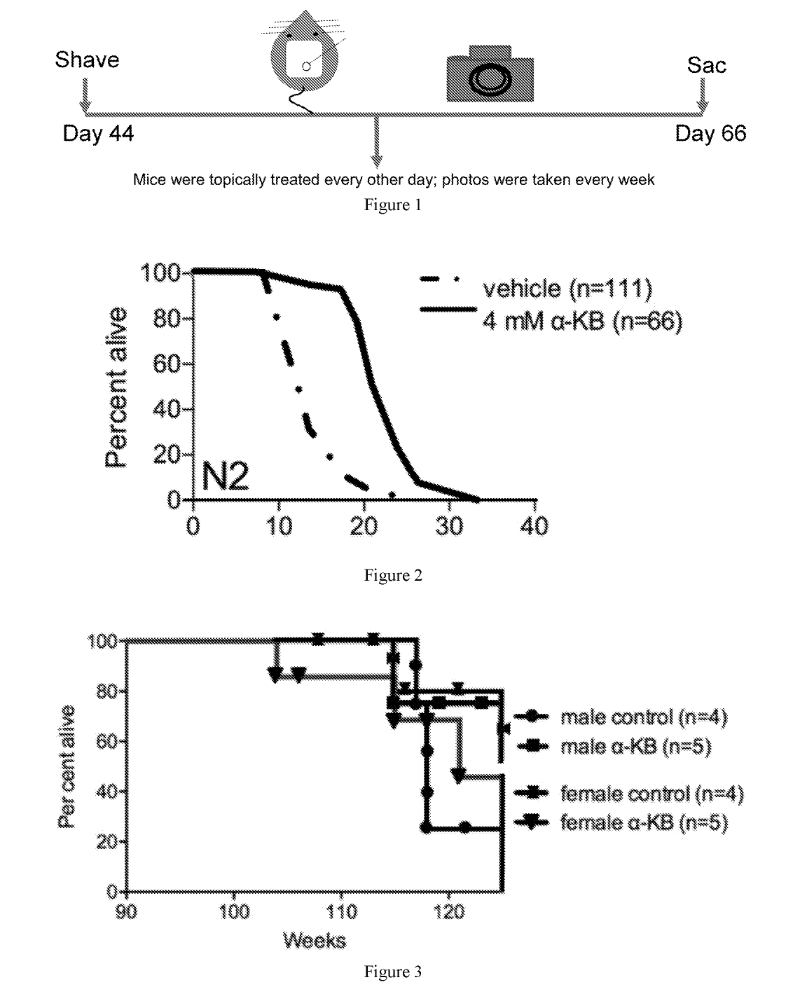

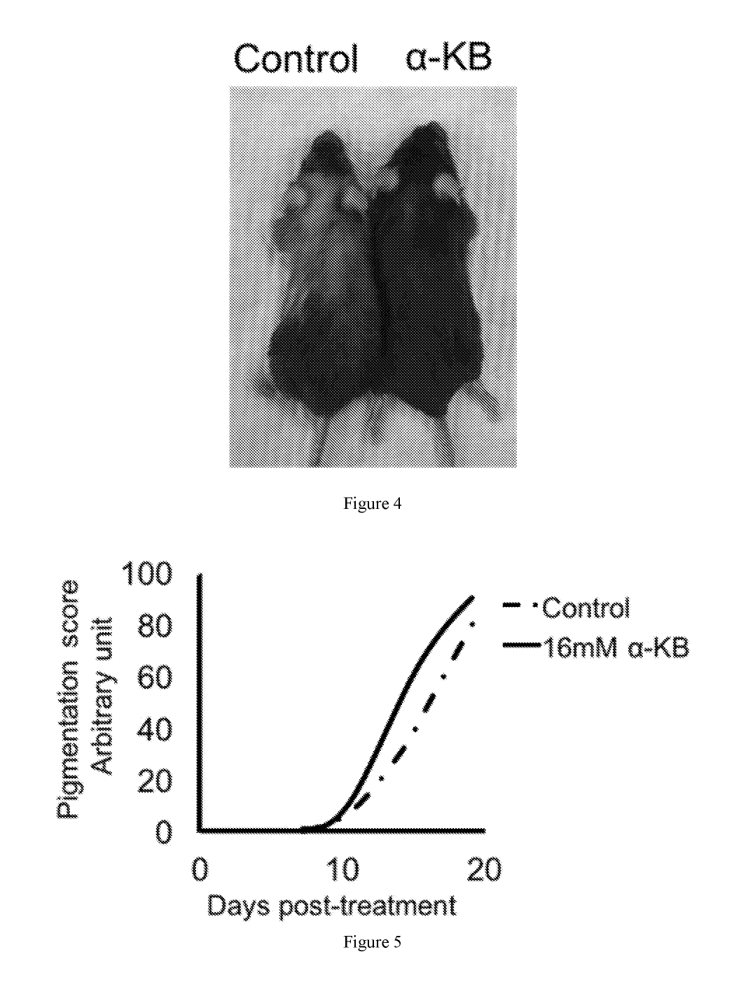

Based on the lifespan extending effects of α-KB in C. elegans (FIG. 2), the anti-aging properties in mice were examined. Aged 101-week old C57BL/6J mice were treated with 8 mM α-KB acid dissolved in drinking water for up to 30 weeks. Administration of α-KB prevented aging traits in aged mice (such as cataracts) and significantly increased survival to beyond 131 weeks of age in male animals (*P=0.0476) (FIG. 3). Remarkably, the hair on dorsal skin turned gray and became thinner (bald) during aging in the control group, while α-KB treated mice exhibited black and thick hair (FIG. 4).

Hair loss is a common problem in the human population and can be result from a variety of stresses. Our finding suggests that α-KB could delay the aging of skin, especially aging related hair loss. In some instances, it was found that α-KB treatment stimulated hair growth in young mice, regardless of age.

### Administration of α-KB and Two Other Anti-Aging Compounds, 2-HB and α-KG, Each Accelerates Hair Growth in Young Mice

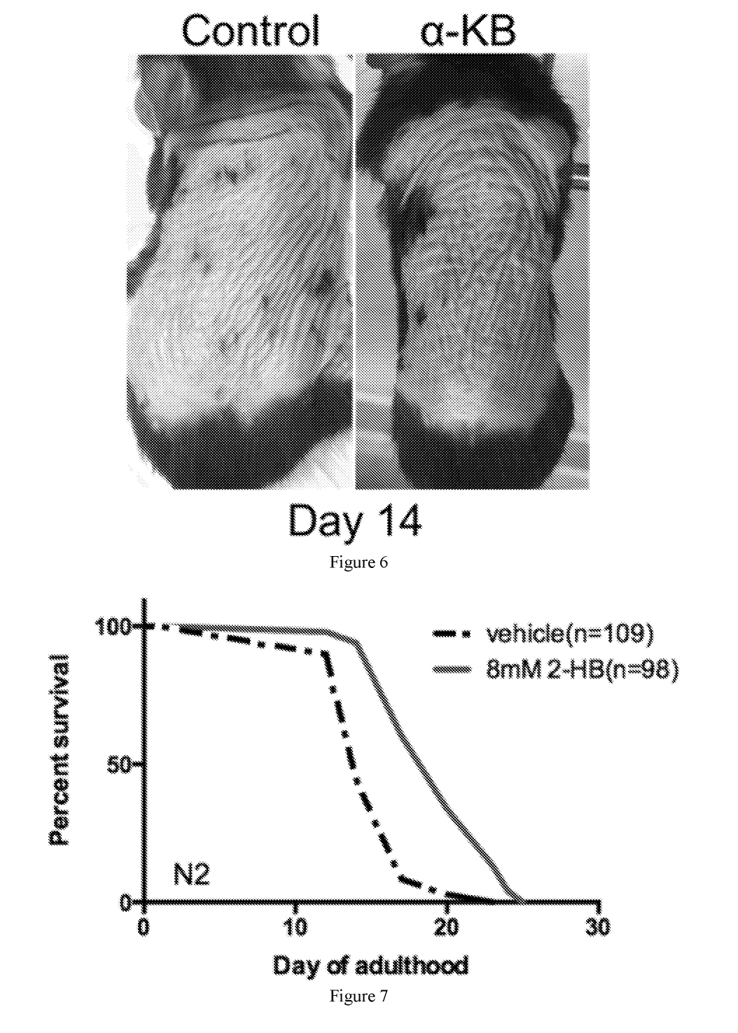

The role α-KB has on stimulating hair growth in 6-week old mice was examined. Dorsal hair was shaved by trimmer at Week 7 and α-KB acid was dissolved in Transdermal Gel at 16 mM and applied on mice dorsal skin every other day. The treated skin started to show pigmentation patches after one week (FIG. 5) and their hair growth was accelerated by α-KB treatment, compared to control (FIG. 6).

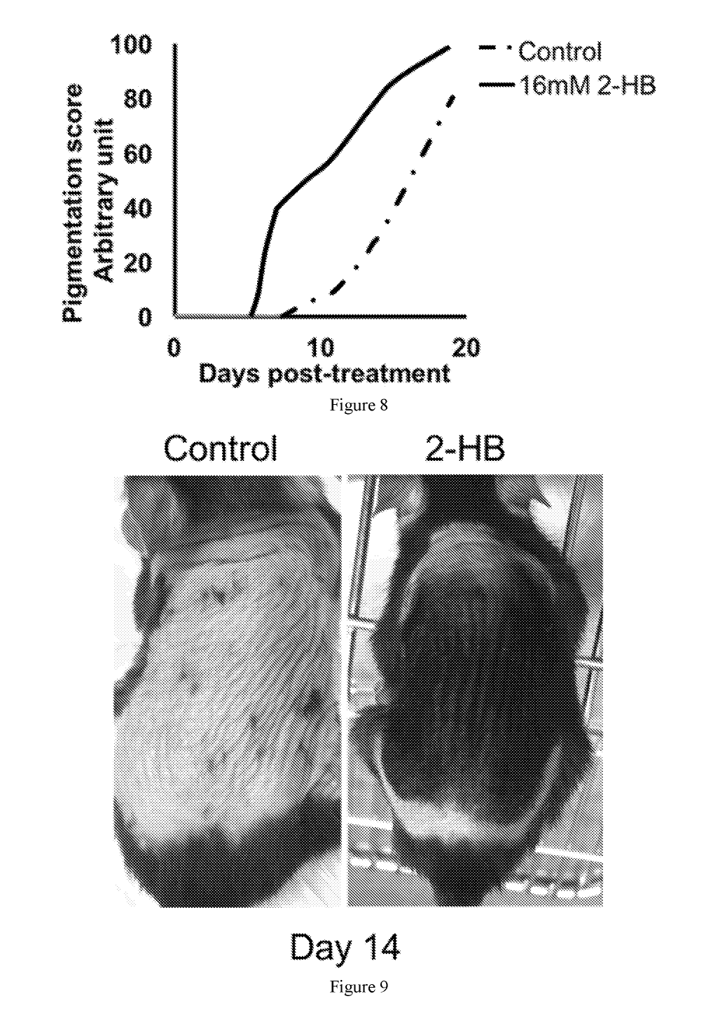

In addition to α-KB, its product from lactate dehydrogenase, 2-HB, can not only extend lifespan of C. elegans (FIG. 7) but also expedite hair growth in young mice (not yet tested in old mice). Upon shaving at Week 7, the mice were topically treated with 16 mM 2-HB every other day. Similarly, 2-HB treated dorsal skin showed pigmentation earlier (FIG. 8) and had hair growing back faster (FIG. 9). Therefore, 2-HB, as an anti-aging compound, can also be used as activator for hair growth.

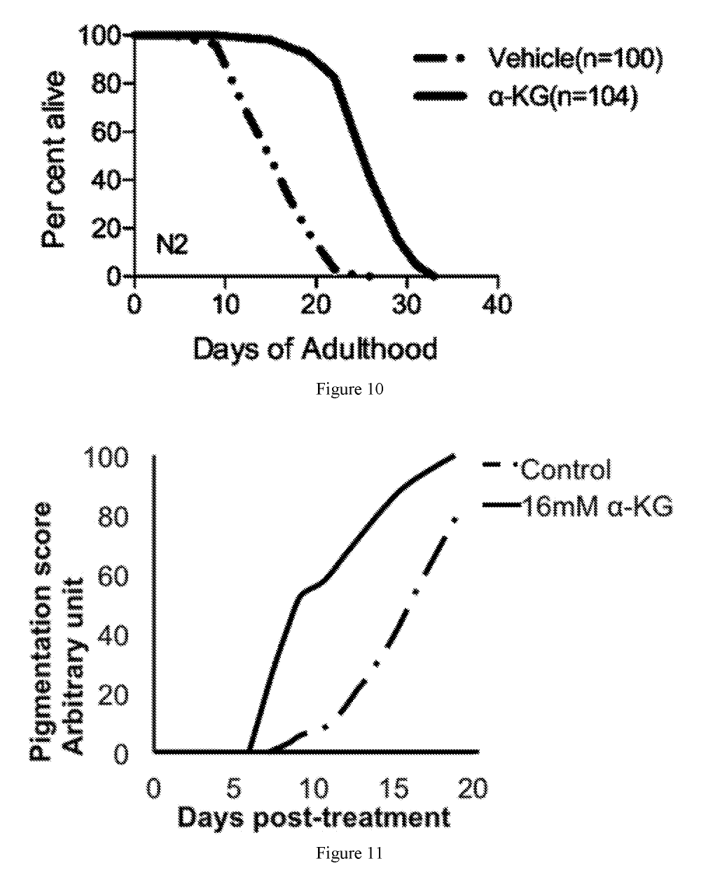

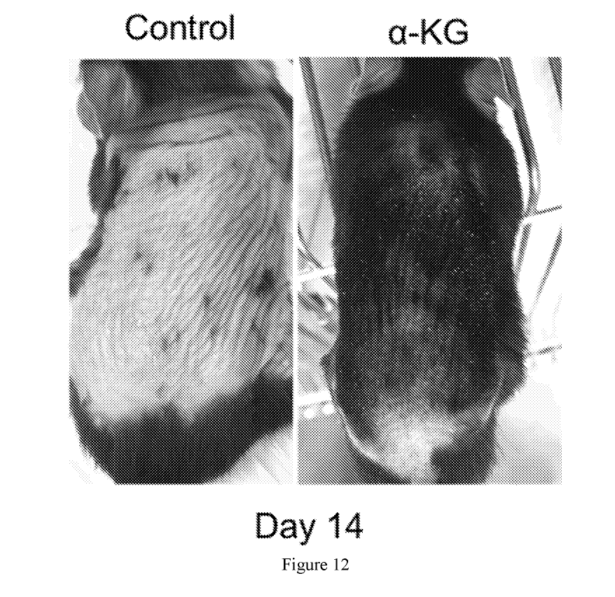

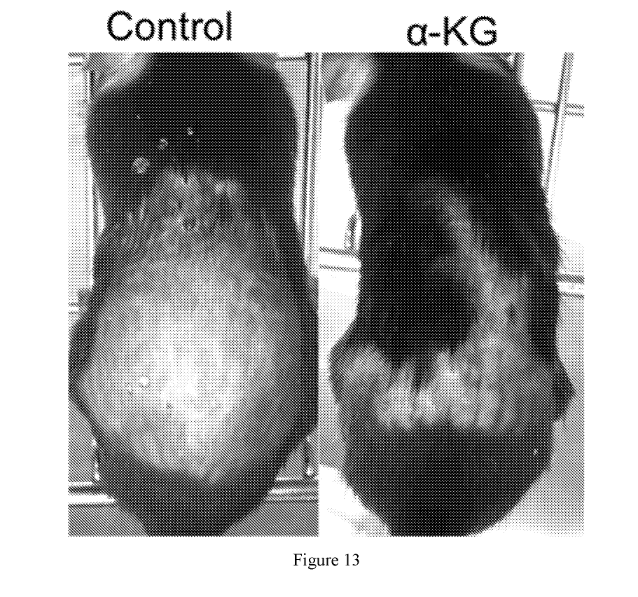

Previously, it was shown that α-KG can extend lifespan in C. elegans by inhibiting mitochondrial complex V (FIG. 10; and Chin et al., Nature 2014). In some instances, this anti-aging compound also accelerated hair growth in young mice. Shaved dorsal skin was treated with 16 mM α-KG and pigmentation burst was observed after one week (FIG. 11). The treated skin had longer and thicker hair than control under a two weeks application (FIG. 12). Mice were also treated with α-KG in drinking water and confirmed its effects for hair growth (FIG. 13)

In some cases, α-KB, α-KG, and 2-HB were demonstrated to stimulate hair growth and improve pigmentation in subjects.

### SPECIFIC EMBODIMENTS

### Embodiment 1

A method for treating, inhibiting, or reducing hair loss in a subject which comprises administering to the subject a therapeutically effective amount of one or more one or more α-ketobutyrate compounds and/or one or more glutarate compounds.

### Embodiment 2

A method for improving or stimulating hair growth in a subject which comprises administering to the subject a therapeutically effective amount of one or more α-ketobutyrate compounds and/or one or more glutarate compounds.

### Embodiment 3

A method for treating, inhibiting, or reducing pigmentation loss in a subject which comprises administering to the subject a therapeutically effective amount of one or more α-ketobutyrate compounds and/or one or more glutarate compounds.

### Embodiment 4

A method for improving or stimulating pigmentation production in a subject which comprises administering to the subject a therapeutically effective amount of one or more α-ketobutyrate compounds and/or one or more glutarate compounds.

### Embodiment 5

The method of Embodiment 1, wherein the hair loss is a result of the subject aging.

### Embodiment 6

The method of Embodiment 3, wherein the pigmentation loss is a result of the subject aging.

### Embodiment 7

The method of any one of the embodiments of Embodiment 1 to Embodiment 6, wherein the subject is aging and/or the subject is an aged subject.

### Embodiment 8

The method of any one of the embodiments of Embodiment 1 to Embodiment 7, wherein the therapeutically effective amount is administered as several doses over a given period of time, e.g., a daily dose for a week or more.

### Embodiment 9

The method of any one of the embodiments of Embodiment 1 to Embodiment 7, wherein the therapeutically effective amount is administered as a daily dose of about 0.01-1.0, preferably about 0.01-0.5, more preferably about 0.1-0.2 grams per kilogram body weight per day.

### Embodiment 10

The method of any one of the embodiments of Embodiment 1 to Embodiment 7, wherein about 0.05 to about 2 grams of the one or more α-ketobutyrate compounds and/or the one or more glutarate compounds per kilogram weight of the subject is administered to the subject daily for at least a week.

### Embodiment 11

The method of any one of the embodiments of Embodiment 1 to Embodiment 10, wherein the one or more α-ketobutyrate compounds is α-ketobutyrate (α-KB), the one or more glutarate compounds is α-ketoglutarate (α-KG), and/or the one or more glutarate compounds is 2-hydroxypentanedioate (2-HG).

### Embodiment 12

An α-ketobutyrate compound and/or a glutarate compound for use in treating, inhibiting, or reducing hair loss, treating, inhibiting, or reducing pigmentation loss, improving, or stimulating hair growth, and/or improving or stimulating pigmentation production in a subject.

### Embodiment 13

A method of stimulating new hair growth in a subject in need thereof, comprising: administering to the subject a pharmaceutical composition comprising a therapeutically effective amount of a compound of Formula I:

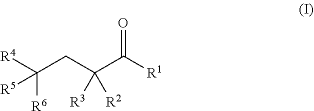

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof; and an excipient; and wherein the pharmaceutical composition is administered to an area on the subject absent of hair to stimulate new hair growth.

### Embodiment 14

The method of Embodiment 13, wherein R¹ is hydrogen, —CHO, or —OR⁷.

### Embodiment 15

The method of Embodiment 14, wherein R¹ is —OR⁷, wherein R⁷ is hydrogen, substituted or unsubstituted alkyl, or substituted or unsubstituted aryl.

### Embodiment 16

The method of Embodiment 15, wherein R¹ is —OR⁷, wherein R⁷ is C₁₋₂₀ substituted or unsubstituted alkyl.

### Embodiment 17

The method of Embodiment 13, wherein R² is hydrogen, halogen, —CN, —CHO, or —NR⁸R⁹, wherein Re and R⁹ are each independently hydrogen or substituted or unsubstituted alkyl.

### Embodiment 18

The method of Embodiment 13, wherein R² and R³, together with the atom to which they are bound, form an oxo.

### Embodiment 19

The method of Embodiment 13, wherein R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl.

### Embodiment 20

The method of Embodiment 19, wherein R⁴ is —COOR⁷ or —CONR⁸R⁹, wherein R⁷, Re, and R⁹ are each independently hydrogen or C₁₋₂₀ substituted or unsubstituted alkyl.

### Embodiment 21

The method of Embodiment 13, wherein the area is absent of hair due to a disease or condition that decreases or inhibits hair growth.

### Embodiment 22

The method of Embodiment 13 or Embodiment 21, wherein the area is absent of hair due to an injury.

### Embodiment 23

The method of Embodiment 13 or Embodiment 21, wherein the area is absent of hair due to chemotherapy and/or radiation therapy.

### Embodiment 24

The method of Embodiment 13 or Embodiment 21, wherein the area is absent of hair due to surgery.

### Embodiment 25

The method of Embodiment 13, wherein the subject has a thyroid disorder.

### Embodiment 26

The method of Embodiment 13, wherein the subject has a pituitary gland disorder.

### Embodiment 27

The method of Embodiment 13, wherein the subject has alopecia areata.

### Embodiment 28

The method of Embodiment 13, wherein the subject has anagen effluvium and/or telogen effluvium.

### Embodiment 29

The method of Embodiment 13, wherein the compound of Formula I is α-ketoglutarate (α-KG).

### Embodiment 30

The method of Embodiment 13, wherein the compound of Formula I is 2-HB.

### Embodiment 31

The method of Embodiment 13, wherein the compound of Formula I is α-ketobutyrate (α-KB).

### Embodiment 32

The method of Embodiment 13 or Embodiment 29, wherein the concentration of α-KG is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 33

The method of Embodiment 32, wherein the concentration of α-KG is about 16 mM.

### Embodiment 34

The method of Embodiment 13 or Embodiment 31, wherein the concentration of α-KB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 35

The method of Embodiment 34, wherein the concentration of α-KB is about 8 mM.

### Embodiment 36

The method of Embodiment 1 or Embodiment 30, wherein the concentration of 2-HB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 37

The method of any one of the embodiments of Embodiment 13 to Embodiment 36, wherein the compound of Formula I is formulated for oral, parenteral, or topical administration.

### Embodiment 38

The method of any one of the embodiments of Embodiment 13 to Embodiment 37, wherein the compound of Formula I is formulated for topical administration.

### Embodiment 39

The method of Embodiment 38, wherein the compound of Formula I is formulated as a gel.

### Embodiment 40

The method of Embodiment 38, wherein the compound of Formula I is formulated as a cream.

### Embodiment 41

The method of Embodiment 38, wherein the compound of Formula I is formulated as an ointment.

### Embodiment 42

The method of Embodiment 38, wherein the compound of Formula I is formulated as a paste.

### Embodiment 43

The method of Embodiment 38, wherein the compound of Formula I is formulated as a lotion.

### Embodiment 44

The method of any one of the embodiments of Embodiment 13 to Embodiment 43, wherein the therapeutically effective amount is administered as a single dose.

### Embodiment 45

The method of any one of the embodiments of Embodiment 13 to Embodiment 43, wherein the therapeutically effective amount is administered in at least two doses, at least three doses, at least four doses, at least five doses, or more.

### Embodiment 46

The method of any one of the embodiments of Embodiment 13 to Embodiment 45, wherein the therapeutically effective amount is administered daily.

### Embodiment 47

The method of any one of the embodiments of Embodiment 13 to Embodiment 45, wherein the therapeutically effective amount is administered every other day.

### Embodiment 48

The method of any one of the embodiments of Embodiment 13 to Embodiment 47, wherein the method further comprises administering to the subject an additional agent.

### Embodiment 49

The method of Embodiment 48, wherein the additional agent comprises one or more growth factors.

### Embodiment 50

The method of Embodiment 49, wherein the growth factor comprises TGF-β2, IGF-1, KGF or HGF.

### Embodiment 51

The method of any one of the embodiments of Embodiment 48 to Embodiment 50, wherein the additional agent is administered in combination with the pharmaceutical composition.

### Embodiment 52

The method of any one of the embodiments of Embodiment 48 to Embodiment 50, wherein the additional agent is administered sequentially with the pharmaceutical composition.

### Embodiment 53

The method of any one of the embodiments of Embodiment 48 to Embodiment 51, wherein the additional agent and the pharmaceutical composition are administered as a unified dosage form.

### Embodiment 54

The method of any one of the embodiments of Embodiment 48 to Embodiment 50 or Embodiment 52, wherein the additional agent and the pharmaceutical composition are administered as separate dosage forms.

### Embodiment 55

The method of any one of the embodiments of Embodiment 13 to Embodiment 54, wherein the number of hair follicles in the subject after administration of the pharmaceutical composition is higher relative to the number of hair follicles in the subject prior to administration of the pharmaceutical composition. In some embodiments, the increase in the number of hair follicles is observed after 1 week of treatment at least every other day. In some embodiments, the increase in the number of hair follicles is observed after 2 weeks of treatment at least every other day.

### Embodiment 56

The method of any one of the embodiments of Embodiment 13 to Embodiment 55, wherein the weight of a hair in the subject after administration of the pharmaceutical composition is greater relative to the weight of a hair in the subject prior to administration of the pharmaceutical composition. In some embodiments, the increase in the weight of the hair is observed after 1 week of treatment at least every other day. In some embodiments, the increase in the weight of the hair is observed after 2 weeks of treatment at least every other day.

### Embodiment 57

The method of any one of the embodiments of Embodiment 13 to Embodiment 56, wherein the hair shaft length of a hair in the subject is increased faster after administration of the pharmaceutical composition relative to the hair shaft length of a hair in the subject prior to administration of the pharmaceutical composition. In some embodiments, the increase in the hair shaft length is observed after 1 week of treatment at least every other day. In some embodiments, the increase in the hair shaft length is observed after 2 weeks of treatment at least every other day.

### Embodiment 58

The method of any one of the embodiments of Embodiment 13 to Embodiment 57, wherein the growth rate of a hair in the subject is increased after administration of the pharmaceutical composition relative to the growth rate of a hair in the subject prior to administration of the pharmaceutical composition. In some embodiments, the increase in the growth rate is observed after 1 week of treatment at least every other day. In some embodiments, the increase in the growth rate is observed after 2 weeks of treatment at least every other day.

### Embodiment 59

The method of Embodiment 1, wherein the subject is a human.

### Embodiment 60

A dosage form comprising a compound of Formula I:

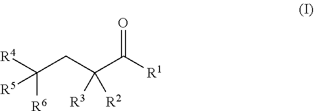

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof;
and an excipient.

### Embodiment 61

The dosage form of Embodiment 60, wherein R¹ is hydrogen, —CHO, or —OR⁷.

### Embodiment 62

The dosage form of Embodiment 61, wherein R¹ is —OR⁷, wherein R⁷ is hydrogen, substituted or unsubstituted alkyl, or substituted or unsubstituted aryl.

### Embodiment 63

The dosage form of Embodiment 62, wherein R¹ is —OR⁷, wherein R⁷ is C₁₋₂₀ substituted or unsubstituted alkyl.

### Embodiment 64

The dosage form of Embodiment 60, wherein R² is hydrogen, halogen, —CN, —CHO, or —NR⁸R⁹, wherein R⁸ and R⁹ are each independently hydrogen or substituted or unsubstituted alkyl.

### Embodiment 65

The dosage form of Embodiment 60, wherein R² and R³, together with the atom to which they are bound, form an oxo.

### Embodiment 66

The dosage form of Embodiment 60, wherein R⁴, R⁵, and R⁶ are each independently hydrogen, —CHO, —OR⁷, —NR⁸R⁹, —COOR⁷, or —CONR⁸R⁹, wherein R⁷, Re, and R⁹ are each independently hydrogen or C₁₋₂₀ substituted or unsubstituted alkyl.

### Embodiment 67

The dosage form of Embodiment 66, wherein R⁴ is —COOR⁷ or —CONR⁸R⁹, wherein R⁷, Re, and R⁹ are each independently hydrogen or C₁₋₂₀ substituted or unsubstituted alkyl.

### Embodiment 68

The dosage form of Embodiment 60, wherein the dosage form is formulated for stimulating a cell to enter into anagen phase.

### Embodiment 69

The dosage form of Embodiment 60, wherein the compound of Formula I is α-ketoglutarate (α-KG).

### Embodiment 70

The dosage form of Embodiment 60, wherein the compound of Formula I is 2-HB.

### Embodiment 71

The dosage form of Embodiment 60, wherein the compound of Formula I is α-ketobutyrate (α-KB).

### Embodiment 72

The dosage form of Embodiment 60 or Embodiment 69, wherein the concentration of α-KG is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 73

The dosage form of Embodiment 72, wherein the concentration of α-KG is about 16 mM.

### Embodiment 74

The dosage form of Embodiment 60 or Embodiment 71, wherein the concentration of α-KB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 75

The dosage form of Embodiment 74, wherein the concentration of α-KB is about 8 mM.

### Embodiment 76

The dosage form of Embodiment 60 or Embodiment 70, wherein the concentration of 2-HB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 77

The dosage form of any one of the embodiments of Embodiment 60 to Embodiment 76, wherein the dosage form is formulated for oral, parenteral, or topical administration.

### Embodiment 78

The dosage form of any one of the embodiments of Embodiment 60 to Embodiment 77, wherein the dosage form is formulated for topical administration.

### Embodiment 79

The dosage form of Embodiment 78, wherein the dosage form is formulated as a gel.

### Embodiment 80

The dosage form of Embodiment 78, wherein the dosage form is formulated as a cream.

### Embodiment 81

The dosage form of Embodiment 78, wherein the dosage form is formulated as an ointment.

### Embodiment 82

The dosage form of Embodiment 78, wherein the dosage form is formulated as a paste.

### Embodiment 83

The dosage form of Embodiment 78, wherein the dosage form is formulated as a lotion.

### Embodiment 84

The dosage form of any one of the embodiments of Embodiment 60 to Embodiment 83, wherein the dosage form is administered as a single dose.

### Embodiment 85

The dosage form of any one of the embodiments of Embodiment 60 to Embodiment 83, wherein the dosage form is administered in at least two doses, at least three doses, at least four doses, at least five doses, or more.

### Embodiment 86

The dosage form of any one of the embodiments of Embodiment 60 to Embodiment 85, wherein the dosage form is administered daily.

### Embodiment 87

The dosage form of any one of the embodiments of Embodiment 60 to Embodiment 85, wherein the dosage form is administered every other day.

### Embodiment 88

The dosage form of any one of the embodiments of Embodiment 60 to Embodiment 87, further comprising an additional agent.

### Embodiment 89

The dosage form of Embodiment 88, wherein the additional agent comprises one or more growth factors.

### Embodiment 90

The dosage form of Embodiment 89, wherein the growth factor comprises TGF-β2, IGF-1, KGF or HGF.

### Embodiment 91

The dosage form of any one of the embodiments of Embodiment 88 to Embodiment 90, wherein the additional agent is administered in combination with the dosage form.

### Embodiment 92

The dosage form of any one of the embodiments of Embodiment 88 to Embodiment 90, wherein the additional agent is administered sequentially with the dosage form.

### Embodiment 93

A topical pharmaceutical composition comprising a compound of Formula I:

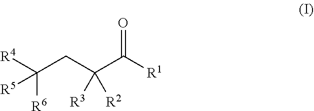

wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof; and
a tissue penetrating enhancer.

### Embodiment 94

The topical pharmaceutical composition of Embodiment 93, wherein R¹ is hydrogen, —CHO, or —OR⁷.

### Embodiment 95

The topical pharmaceutical composition of Embodiment 94, wherein R¹ is —OR⁷, wherein R⁷ is hydrogen, substituted or unsubstituted alkyl, or substituted or unsubstituted aryl.

### Embodiment 96

The topical pharmaceutical composition of Embodiment 95, wherein R¹ is —OR⁷, wherein R⁷ is C₁₋₂₀ substituted or unsubstituted alkyl.

### Embodiment 97

The topical pharmaceutical composition of Embodiment 93, wherein R² is hydrogen, halogen, —CN, —CHO, or —NR⁸R⁹, wherein R⁸ and R⁹ are each independently hydrogen or substituted or unsubstituted alkyl.

### Embodiment 98

The topical pharmaceutical composition of Embodiment 93, wherein R² and R³, together with the atom to which they are bound, form an oxo.

### Embodiment 99

The topical pharmaceutical composition of Embodiment 93, wherein R⁴, R⁵, and R⁶ are each independently hydrogen, —CHO, —OR⁷, —NR⁸R⁹, —COOR⁷, or —CONR⁸R⁹, wherein R⁷, Re, and R⁹ are each independently hydrogen or C1-20 substituted or unsubstituted alkyl.

### Embodiment 100

The topical pharmaceutical composition of Embodiment 99, wherein R⁴ is —COOR⁷ or —CONR⁸R⁹, wherein R⁷, Re, and R⁹ are each independently hydrogen or C₁₋₂₀ substituted or unsubstituted alkyl.

### Embodiment 101

The topical pharmaceutical composition of Embodiment 93, wherein the compound of Formula I is α-ketoglutarate (α-KG).

### Embodiment 102

The topical pharmaceutical composition of Embodiment 93, wherein the compound of Formula I is 2-HB.

### Embodiment 103

The topical pharmaceutical composition of Embodiment 93, wherein the compound of Formula I is α-ketobutyrate (α-KB).

### Embodiment 104

The topical pharmaceutical composition of Embodiment 93 or Embodiment 101, wherein the concentration of α-KG is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 105

The topical pharmaceutical composition of Embodiment 104, wherein the concentration of α-KG is about 16 mM.

### Embodiment 106

The topical pharmaceutical composition of Embodiment 93 or Embodiment 103, wherein the concentration of α-KB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 107

The topical pharmaceutical composition of Embodiment 106, wherein the concentration of α-KB is about 8 mM.

### Embodiment 108

The topical pharmaceutical composition of Embodiment 93 or Embodiment 102, wherein the concentration of 2-HB is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

### Embodiment 109

The topical pharmaceutical composition of any one of the embodiments of Embodiment 93 to Embodiment 108, wherein the topical pharmaceutical composition is formulated as a gel.

### Embodiment 110

The topical pharmaceutical composition of any one of the embodiments of Embodiment 93 to Embodiment 108, wherein the topical pharmaceutical composition is formulated as a cream.

### Embodiment 111

The topical pharmaceutical composition of any one of the embodiments of Embodiment 93 to Embodiment 108, wherein the topical pharmaceutical composition is formulated as an ointment.

### Embodiment 112

The topical pharmaceutical composition of any one of the embodiments of Embodiment 93 to Embodiment 108, wherein the topical pharmaceutical composition is formulated as a paste.

### Embodiment 113

The topical pharmaceutical composition of any one of the embodiments of Embodiment 93 to Embodiment 108, wherein the topical pharmaceutical composition is formulated as a lotion.

All scientific and technical terms used in this application have meanings commonly used in the art unless otherwise specified.

The section headings used herein are for organizational purposes and are not to be construed as limiting the subject matter described.

As used herein, the term “subject” includes humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

To the extent necessary to understand or complete the disclosure of the present invention, all publications, patents, and patent applications mentioned herein are expressly incorporated by reference therein to the same extent as though each were individually so incorporated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method for treating, inhibiting, or reducing hair loss; improving or stimulating hair growth; treating, inhibiting, or reducing pigmentation loss; and/or improving or stimulating pigmentation production in a subject which comprises administering to the subject a therapeutically effective amount of one or more alpha-ketobutyrate compounds and/or one or more glutarate compounds.

2. The method according to claim 1, wherein the one or more alpha-ketobutyrate compounds is alpha-ketobutyrate (α-KB), the one or more glutarate compounds is alpha-ketoglutarate (α-KG), and/or the one or more glutarate compounds is 2-hydroxypentanedioate (2-HG).

3. A method of stimulating new hair growth in a subject in need thereof, comprising: administering to the subject a composition comprising a therapeutically effective amount of a compound of Formula I: 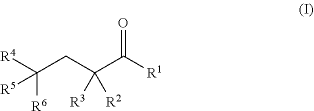
wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof, and
an excipient; and
wherein the composition is administered to an area on the subject absent of hair to stimulate new hair growth.

4. A composition comprising a compound of Formula I: 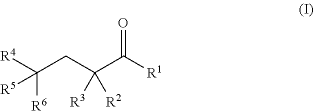
wherein
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; and
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof;
and a pharmaceutically acceptable excipient and/or a tissue penetrating enhancer.

5. The composition according to claim 4, wherein R¹ is
(a) hydrogen, —CHO, or —OR⁷;
(b) —OR⁷, wherein R⁷ is hydrogen, substituted or unsubstituted alkyl, or substituted or unsubstituted aryl; or
(c) —OR⁷, wherein R⁷ is C₁₋₂₀ substituted or unsubstituted alkyl.

6. The composition according to claim 4, wherein
(a) R² is hydrogen, halogen, —CN, —CHO, or —NR⁸R⁹, wherein R⁸ and R⁹ are each independently hydrogen or substituted or unsubstituted alkyl; or
(b) R² and R³, together with the atom to which they are bound, form an oxo.

7. The composition according to claim 4, wherein R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl.

8. The composition according to claim 4, wherein R⁴ is —COOR⁷ or —CONR⁸R⁹, wherein R⁷, R⁸, and R⁹ are each independently hydrogen or C₁₋₂₀ substituted or unsubstituted alkyl.

9. The composition according to claim 4, wherein the compound of Formula I is alpha-ketoglutarate (α-KG), 2-HB, or alpha-ketobutyrate (α-KB).

10. The composition according to claim 4, wherein the concentration of the compound of Formula I in the composition is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, or 50 mM.

11. The composition according to claim 4, wherein the composition is formulated for oral, parenteral, or topical administration.

12. The composition according to claim 4, wherein the composition is formulated as a gel, a cream, an ointment, a paste, or a lotion.

13. The composition according to claim 4, wherein the composition further comprises one or more growth factors, such as TGF-β2, IGF-1, KGF, and HGF.

14. The composition according to claim 4, the composition is capable of stimulating a cell to enter into anagen phase when contacted therewith.

15. The method of claim 3, wherein the area is absent of hair due to a disease or condition that decreases or inhibits hair growth, an injury, chemotherapy and/or radiation therapy, or surgery.

16. The method of claim 3, wherein the subject has a thyroid disorder, a pituitary gland disorder, alopecia areata, anagen effluvium, and/or telogen effluvium.

17. The method of claim 3, wherein the number of hair follicles in the subject after administration of the composition is higher relative to the number of hair follicles in the subject prior to administration of the composition.

18. The method of claim 3, wherein the weight of a hair in the subject after administration of the composition is greater relative to the weight of a hair in the subject prior to administration of the composition.

19. The method of claim 3, wherein the hair shaft length of a hair in the subject is increased faster after administration of the composition relative to the hair shaft length of a hair in the subject prior to administration of the composition.

20. The method of claim 3, wherein the growth rate of a hair in the subject is increased after administration of the composition relative to the growth rate of a hair in the subject prior to administration of the composition.
